(19) United States
(12) Patent Application Publication
(10) Pub. No.: US 2025/0382242 A1
(43) Pub. Date: Dec. 18, 2025

(54) C20 TRISUBSTITUTED OLEFINS PRODUCED BY ACIDIC CATALYZED REACTION OF C20 2-SUBSTITUTED ALPHA OLEFINS

(71) Applicant: Chevron Phillips Chemical Company LP, The Woodlands, TX (US)

(72) Inventors: Jeffery C. Gee, Kingwood, TX (US); Julie A. Leseberg, Kingwood, TX (US); Steven M. Bischof, Spring, TX (US); Thomas J. Malinski, Porter, TX (US); Michael S. Webster-Gardiner, Humble, TX (US); James Hillier, Porter, TX (US); Spencer A. Kerns, Houston, TX (US)

(21) Appl. No.: 18/745,676

(22) Filed: Jun. 17, 2024

(51) Int. Cl. C07C 2/34; C07C 2/12; C07C 2/14; C07C 2/24; C07C 5/03

(52) U.S. Cl. CPC C07C 2/34; C07C 2/12; C07C 2/14; C07C 2/24; C07C 5/03; C07C 2527/135; C07C 2529/06; C07C 2531/22

(57) ABSTRACT

C₂₀ trisubstituted olefins are produced from C₂₀ 2-substituted alpha olefins, which are produced from branched C₁₀ olefins.

### FIELD OF THE DISCLOSURE

The present disclosure generally relates to C₂₀ trisubstituted olefins that are derived from C₂₀ 2-substituted alpha olefins, which are derived from branched C₁₀ olefins.

### BACKGROUND

Olefin oligomerization reactions produce olefins using various catalyst systems that can be used to direct reactions to particular oligomer products. For example, aluminum, nickel, zirconium, and iron based catalyst systems can be used for the synthesis of oligomer products containing C₄ to C₃₀₊ alpha olefins from ethylene. Chromium based catalyst systems can be used for the selective synthesis of oligomer products containing 1-hexene and/or 1-octene from ethylene. Many applications exist for these oligomer products, including employment as intermediates in the manufacture of detergents, as more environmentally friendly replacements where refined oils might otherwise be used, as monomers or comonomers in the production of polyolefins (e.g., polyethylene), and as intermediates for many other types of products. However, the olefin oligomerization reactions can produce by-products which can be removed from the desired oligomerization product.

The oligomerization by-products that are branched C₁₀ olefins can have value as intermediate chemicals for the synthesis of other higher value products. For example, as disclosed in U.S. Pat. No. 11,174,205, branched C₁₀ olefins can be contacted with a dimerization catalyst to from a reaction product that includes C₂₀ 2-substituted alpha olefins. The C₂₀ 2-substituted alpha olefins in U.S. Pat. No. 11,174,205 can be used in various applications, such as a feedstock for making alkenyl succinic anhydride (e.g., via reaction with maleic anhydride), which can be used as a paper sizing agent, or as a lube oil additive, among other uses.

There is ongoing effort to find other uses for the C₂₀ 2-substituted alpha olefins that were synthesized in U.S. Pat. No. 11,174,205.

### SUMMARY

Disclosed is a process including: contacting a C₂₀ 2-substituted alpha olefin composition with an acidic catalyst to form a C₂₀ trisubstituted olefin composition including a C₂₀ trisubstituted olefin.

Disclosed is a process including: contacting a branched C₁₀ olefin composition with a dimerization catalyst or a dimerization catalyst system to form a C₂₀ 2-substituted alpha olefin composition; and contacting the C₂₀ 2-substituted alpha olefin composition with an acidic catalyst to form a C₂₀ trisubstituted olefin composition including a C₂₀ trisubstituted olefin.

Disclosed is a composition including C₂₀ trisubstituted olefins, which can include 5-propyl-6-methyl-9-propyl-tridec-5-ene, 5-propyl-6-methyl-9-propyl-tridec-6-ene, 4-butyl-7-methyl-10-methyl-tetradec-6-ene, 4-butyl-7-methyl-10-methyl-tetradec-7-ene, 5-propyl-6-methyl-10-ethyl-tetradec-5-ene, 5-propyl-6-methyl-10-ethyl-tetradec-6-ene, 5-ethyl-7-methyl-10-propyl-tetradec-6-ene, 5-ethyl-7-methyl-10-propyl-tetradec-7-ene, 5-ethyl-7-methyl-11-ethyl-pentadec-6-ene, 5-ethyl-7-methyl-11-ethyl-pentadec-7-ene, 5-propyl-6-methyl-11-methyl-pentadec-5-ene, 5-propyl-6-methyl-11-methyl-pentadec-6-ene, 5-methyl-8-methyl-12-ethyl-hexadec-7-ene, 5-methyl-8-methyl-12-ethyl-hexadec-8-ene, 5-ethyl-7-methyl-12-methyl-hexadec-6-ene, 5-ethyl-7-methyl-12-methyl-hexadec-7-ene, 5-methyl-8-methyl-13-methyl-heptadec-7-ene, 5-methyl-8-methyl-13-methyl-heptadec-8-ene, 5-propyl-6-methyl-hexadec-5-ene, 5-propyl-6-methyl-hexadec-6-ene, 5-propyl-8-methyl-hexadec-7-ene, 5-propyl-8-methyl-hexadec-8-ene, 5-ethyl-7-methyl-heptadec-7-ene, 5-ethyl-7-methyl-heptadec-8-ene, 5-ethyl-9-methyl-heptadec-8-ene, 5-ethyl-9-methyl-heptadec-9-ene, 5-methyl-8-methyl-octadec-7-ene, 5-methyl-8-methyl-octadec-8-ene, 9-methyl-14-methyl-octadec-8-ene, 9-methyl-14-methyl-octadec-9-ene, 9-methyl-nonadec-8-ene, 9-methyl-nonadec-9-ene, or combinations thereof.

Disclosed is a composition including C₂₀ trisubstituted olefins, any of which can include a linear carbon chain having 13 to 17 carbon atoms, wherein the linear carbon chain having 13 to 17 carbon atoms has a first alkyl group on a carbon in a 4 or 5 position of the linear carbon chain, wherein the first alkyl group is a methyl group, an ethyl group, a propyl group, or a butyl group, wherein the linear carbon chain having 13 to 17 carbon atoms has a second alkyl group on a carbon in a 6, 7, or 8 position of the linear carbon chain, wherein the second alkyl group is a methyl group, wherein the linear carbon chain having 13 to 17 carbon atoms has a third alkyl group on a carbon in a 9, 10, 11, 12, or 13 position of the linear carbon chain, wherein the third alkyl group is a methyl group, an ethyl group, or a propyl group, wherein a carbon-carbon double bond is on a carbon in the 5, 6, 7, or 8 position of the linear carbon chain having 13 to 17 carbon atoms.

Disclosed is a composition including C₂₀ trisubstituted olefins, any of which can include a linear carbon chain having 16 to 18 carbon atoms, wherein the linear carbon chain having 16 to 18 carbon atoms has a first alkyl group on a carbon in a 5 or 9 position of the linear carbon chain, wherein the first alkyl group is a methyl group, an ethyl group, or a propyl group, wherein the linear carbon chain having 16 to 18 carbon atoms has a second alkyl group on a carbon in a 6, 7, 8, or 14 position of the linear carbon chain, wherein the second alkyl group is a methyl group, wherein a carbon-carbon double bond is on a carbon in the 5, 6, 7, 8, or 9 position of the linear carbon chain having 16 to 18 carbon atoms.

Disclosed is a composition including C₂₀ trisubstituted olefins, any of which can include a linear carbon chain having 19 carbon atoms, wherein the linear carbon chain having 19 carbon atoms has a first alkyl group on a carbon in a 9 position of the linear carbon chain, wherein the first alkyl group is a methyl group, wherein a carbon-carbon double bond is on a carbon in a 8 or 9 position of the linear carbon chain having 19 carbon atoms.

Other technical features may be readily apparent to one skilled in the art from the following figures, descriptions, and claims.

### DETAILED DESCRIPTION

Illustrative aspects of the subject matter claimed herein will now be disclosed. In the interest of clarity, not all features of an actual implementation are described in this specification. It can be appreciated that in the development of any such actual aspect, numerous implementation-specific decisions must be made to achieve the developers' specific goals, such as compliance with system-related and business-related constraints, which can vary from one implementation to another. Moreover, it can be appreciated that such a development effort, even if complex and time-consuming, would be a routine undertaking for those of ordinary skill in the art having the benefit of this disclosure.

In the description herein, various ranges and/or numerical limitations can be expressly stated below. It should be recognized that unless stated otherwise, it is intended that endpoints are to be interchangeable. Further, any ranges include iterative ranges of like magnitude falling within the expressly stated ranges or limitations.

Furthermore, various modifications can be made within the scope of the invention as herein intended, and aspects of the invention can include combinations of features other than those expressly claimed. In particular, flow arrangements other than those expressly described herein are within the scope of the invention.

Unless otherwise specified, the terms “contact” and “combine,” and their derivatives, can refer to any addition sequence, order, or concentration for contacting or combining two or more components of the disclosed embodiments. Combining or contacting of dimerization components can occur in one or more reaction zones under suitable contact conditions such as temperature, pressure, contact time, flow rates, etc.

Within this specification, the word “reactor” refers to a single piece of equipment, such as, for example, a vessel, in which a reaction takes place, but excludes any associated equipment such as piping, pumps, and the like which is external to the vessel. Examples of reactors include stirred tank reactors (e.g., a continuous stirred tank reactor), plug flow reactors, or any other type of reactor. Within this specification “reaction zone” refers to any portion of equipment in which a desired reaction occurs, including but not limited to, a reactor, associated piping, associated pumps, and any other associated equipment. It should be noted that in some cases a “reactor” can also be a “reaction zone.” The terms “reactor” and “reaction zone” can be qualified to refer to more specific “reactors” and “reaction zones” by use of additional qualifying terms. For example, the use of the term “dimerization reactor” and “dimerization reaction zone” indicates that the desired reaction within the reactor and/or reaction zone is a dimerization reaction.

Within this specification, term “reaction zone” refers to the portion of a process, the associated equipment and associated process lines where all the necessary reaction components and reaction conditions are present such that the reaction can occur at a desired rate. That is to say that the reaction zone begins where the necessary reaction components and reaction conditions are present to maintain the reaction within 25 percent of the average reaction rate and the reaction system ends where the conditions do not maintain a reaction rate within 25 percent of the average reaction rate (based upon a volume average of the reaction rate of the reaction system). For example, in terms of a dimerization process, the reaction zone begins at the point where sufficient feedstock and active catalyst system is present under the sufficient reaction conditions to maintain dimerization product production at the desired rate and the reaction zone ends at a point where either the catalyst system is deactivated, sufficient feedstock is not present to sustain dimerization product production, or other reaction conditions are not sufficient to maintain the dimerization product production or the desired dimerization product production rate. Within this specification the “reaction zone” can comprise one or more reactor zone, one or more reactors, and associated equipment where all the necessary reaction components and reaction conditions are present such that the reaction can occur at a desired rate. The use of the term “dimerization reaction zone” indicates that the desired reaction within the reaction zone is a dimerization reaction.

Unless otherwise indicated, the definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition can be applied. To the extent that any definition or usage provided by any document incorporated herein by reference conflicts with the definition or usage provided herein, the definition or usage provided herein controls.

For any particular compound disclosed herein, the general structure or name presented is also intended to encompass all structural isomers, conformational isomers, and stereoisomers that can arise from a particular set of substituents, unless indicated otherwise. Thus, a general reference to a compound includes all structural isomers unless explicitly indicated otherwise; e.g., a general reference to hexene includes 1-hexene, 2-hexene, 3-hexene, and any other hydrocarbon having 6 carbon atoms (linear, branched, or cyclic) and a single carbon-carbon double bond. Additionally, the reference to a general structure or name encompasses all enantiomers, diastereomers, and other optical isomers whether in enantiomeric or racemic forms, as well as mixtures of stereoisomers, as the context permits or requires. For any particular formula or name that is presented, any general formula or name presented also encompasses all conformational isomers, regioisomers, and stereoisomers that can arise from a particular set of substituents.

A chemical “group” is described according to how that group is formally derived from a reference or “parent” compound, for example, by the number of hydrogen atoms formally removed from the parent compound to generate the group, even if that group is not literally synthesized in this manner. By way of example, an “alkyl group” formally can be derived by removing one hydrogen atom from an alkane, while an “alkylene group” formally can be derived by removing two hydrogen atoms from an alkane. Moreover, a more general term can be used to encompass a variety of groups that formally are derived by removing any number (“one or more”) hydrogen atoms from a parent compound, which in this example can be described as an “alkane group,” and which encompasses an “alkyl group,” an “alkylene group,” and materials have three or more hydrogens atoms, as necessary for the situation, removed from the alkane. Throughout, the disclosure of a substituent, ligand, or other chemical moiety can constitute a particular “group” implies that the well-known rules of chemical structure and bonding are followed when that group is employed as described. When describing a group as being “derived by,” “derived from,” “formed by,” or “formed from,” such terms are used in a formal sense and are not intended to reflect any specific synthetic methods or procedure, unless specified otherwise or the context requires otherwise.

The term “hydrocarbyl group” is used herein in accordance with the definition specified by IUPAC: a univalent group formed by removing a hydrogen atom from a hydrocarbon. Similarly, a “hydrocarbylene group” refers to a group formed by removing two hydrogen atoms from a hydrocarbon, either two hydrogen atoms from one carbon atom or one hydrogen atom from each of two different carbon atoms. Therefore, in accordance with the terminology used herein, a “hydrocarbon group” refers to a generalized group formed by removing one or more hydrogen atoms (as necessary for the particular group) from a hydrocarbon. A “hydrocarbyl group,” “hydrocarbylene group,” and “hydrocarbon group” can be acyclic or cyclic groups, and/or can be linear or branched. A “hydrocarbyl group,” “hydrocarbylene group,” and “hydrocarbon group” can include rings, ring systems, aromatic rings, and aromatic ring systems, which contain only carbon and hydrogen. “Hydrocarbyl groups,” “hydrocarbylene groups,” and “hydrocarbon groups” include, by way of example, aryl, arylene, arene, alkyl, alkylene, alkane, cycloalkyl, cycloalkylene, cycloalkane, aralkyl, aralkylene, and aralkane groups, among other groups, as members.

The term “alkane” whenever used in this specification and claims refers to a saturated hydrocarbon compound. Other identifiers can be utilized to indicate the presence of particular groups in the alkane (e.g., halogenated alkane indicates that the presence of one or more halogen atoms replacing an equivalent number of hydrogen atoms in the alkane). The term “alkyl group” is used herein in accordance with the definition specified by IUPAC: a univalent group formed by removing a hydrogen atom from an alkane. Similarly, an “alkylene group” refers to a group formed by removing two hydrogen atoms from an alkane (either two hydrogen atoms from one carbon atom or one hydrogen atom from two different carbon atoms). An “alkane group” is a general term that refers to a group formed by removing one or more hydrogen atoms (as necessary for the particular group) from an alkane. An “alkyl group,” “alkylene group,” and “alkane group” can be acyclic or cyclic groups, and/or can be linear or branched unless otherwise specified. Primary, secondary, and tertiary alkyl groups are derived by removal of a hydrogen atom from a primary, secondary, or tertiary carbon atom, respectively, of an alkane. The n-alkyl group can be derived by removal of a hydrogen atom from a terminal carbon atom of a linear alkane.

An aliphatic compound is an acyclic or cyclic, saturated or unsaturated carbon compound, excluding aromatic compounds. Thus, an aliphatic compound is an acyclic or cyclic, saturated or unsaturated carbon compound, excluding aromatic compounds; that is, an aliphatic compound is a non-aromatic organic compound. An “aliphatic group” is a generalized group formed by removing one or more hydrogen atoms (as necessary for the particular group) from the carbon atom of an aliphatic compound. Aliphatic compounds and therefore aliphatic groups can contain organic functional group(s) and/or atom(s) other than carbon and hydrogen.

The term “substituted” when used to describe a compound or group, for example, when referring to a substituted analog of a particular compound or group, is intended to describe any non-hydrogen moiety that formally replaces a hydrogen in that group, and is intended to be non-limiting. A group or groups can also be referred to herein as “unsubstituted” or by equivalent terms such as “non-substituted,” which refers to the original group in which a non-hydrogen moiety does not replace a hydrogen within that group. “Substituted” is intended to be non-limiting and include inorganic substituents or organic substituents.

The term “olefin” whenever used in this specification and claims refers to hydrocarbons that have at least one carbon-carbon double bond that is not part of an aromatic ring or an aromatic ring system. The term “olefin” includes aliphatic and aromatic, cyclic and acyclic, and/or linear and branched hydrocarbons having at least one carbon-carbon double bond that is not part of an aromatic ring or ring system unless specifically stated otherwise. Olefins having only one, only two, only three, etc . . . carbon-carbon double bonds can be identified by use of the term “mono,” “di,” “tri,” etc . . . within the name of the olefin. The olefins can be further identified by the position of the carbon-carbon double bond(s).

The term “alkene” whenever used in this specification and claims refers to a linear or branched aliphatic hydrocarbon olefin that has one or more carbon-carbon double bonds. Alkenes having only one, only two, only three, etc . . . such multiple bonds can be identified by use of the term “mono,” “di,” “tri,” etc . . . within the name. Other identifiers can be utilized to indicate the presence or absence of particular groups within an alkene. For example, a haloalkene refers to an alkene having one or more hydrogen atoms replaced with a halogen atom.

The term “alpha olefin” as used in this specification and claims refers to an olefin that has a carbon-carbon double bond between the first and second carbon atoms of the longest contiguous chain of carbon atoms. The term “alpha olefin” includes linear and branched alpha olefins unless expressly stated otherwise. In the case of branched alpha olefins, a branch can be at the 2-position (a vinylidene) and/or the 3-position or higher with respect to the olefin double bond. The term “vinylidene” whenever used in this specification and claims refers to an alpha olefin having a branch at the 2-position with respect to the olefin double bond. By itself, the term “alpha olefin” does not indicate the presence or absence of other carbon-carbon double bonds unless explicitly indicated.

The term “reaction zone effluent,” and it derivatives (e.g., dimerization reaction zone effluent) generally refers to all the material which exits the reaction zone through a reaction zone outlet/discharge which discharges a reaction mixture and can include reaction zone feed(s) (e.g., olefin, catalyst system or catalyst system components, and/or solvent), and/or reaction product (e.g., dimerization product and dimerization by-product). The term “reaction zone effluent” and its derivatives can be qualified to refer to certain portions by use of additional qualifying terms. For example, while reaction zone effluent refers to all material which exits the reaction zone through the reaction zone outlet/discharge, a reaction zone dimerization product effluent refers to only the dimerization product within the reaction zone effluent.

Features within this disclosure that are provided as minimum values can be alternatively stated as “at least” or “greater than or equal to” any recited minimum value for the feature disclosed herein. Features within this disclosure that are provided as maximum values can be alternatively stated as “less than or equal to” any recited maximum value for the feature disclosed herein.

Within this disclosure the normal rules of organic nomenclature prevail. For instance, when referencing substituted compounds or groups, references to substitution patterns are taken to indicate that the indicated group(s) is (are) located at the indicated position and that all other non-indicated positions are hydrogen. For example, reference to a 4-substituted phenyl group indicates that there is a non-hydrogen substituent located at the 4 position and hydrogens located at the 2, 3, 5, and 6 positions. References to compounds or groups having substitution at positions in addition to the indicated position can be referenced using comprising or some other alternative language. For example, a reference to a phenyl group comprising a substituent at the 4 position refers to a phenyl group having a non-hydrogen substituent group at the 4 position and hydrogen or any non-hydrogen group at the 2, 3, 5, and 6 positions.

Processes and/or, methods described herein can utilize steps, features, and compounds which are independently described herein. The process and/or methods described herein may or may not utilize step identifiers (e.g., 1), 2), etc., a), b), etc., i), ii), etc., or first, second etc., among others), features (e.g., 1), 2), etc., a), b), etc., i), ii), etc., or first, second etc., among others), and/or compound and/or composition identifiers (e.g., 1), 2), etc., a), b), etc., i), ii), etc., or first, second etc., among others). However, it should be noted that processes and/or methods described herein can have multiple steps, features (e.g., reagent ratios, formation conditions, among other considerations), and/or multiple compounds and/or composition using no descriptor or sometimes having the same general identifier. Consequently, it should be noted that the processes and/or methods described herein can be modified to use an appropriate step or feature identifier (e.g., 1), 2), etc., a), b), etc., i), ii), etc., or first, second etc., among others), feature identifier features (e.g., 1), 2), etc., a), b), etc., i), ii), etc., or first, second etc., among others), and/or compound identifier (e.g., first, second, etc.) regardless of step, feature, and/or compound identifier utilized in the a particular statement, aspect, and/or embodiment described herein and that step or feature identifiers can be added and/or modified to indicate individual different steps/features/compounds utilized within the process and/or methods without detracting from the general disclosure.

### Introduction

Disclosed herein are compositions having C₂₀ trisubstituted olefins and the processes for producing the compositions. Generally, the processes for producing the compositions comprising the C₂₀ trisubstituted olefins includes 1) contacting a branched C₁₀ olefin composition with a dimerization catalyst or a dimerization catalyst system to form a C₂₀ 2-substituted alpha olefin composition; and 2) contacting the C₂₀ 2-substituted alpha olefin composition with an acidic catalyst to form a C₂₀ trisubstituted olefin composition containing the C₂₀ trisubstituted olefins. The reaction conditions for contacting a branched C₁₀ olefin composition with a dimerization catalyst or a dimerization catalyst system are controlled to promote dimerization of the branched C₁₀ olefins and conversion to the C₂₀ dimers. The reaction conditions for contacting the C₂₀ 2-substituted alpha olefin composition with an acidic catalyst are controlled to promote formation of the C₂₀ trisubstituted olefins and their isomers, while minimizing the formation of any C₄₀ dimers. Valuable uses for the C₂₀ trisubstituted olefins includes use in a feedstock for production of a paper sizing agent or for production of polyalphaolefins. Uses of the C₂₀ trisubstituted olefins disclosed herein are not limited to those uses disclosed herein.

### C₁₀ Olefin Composition

In an aspect, the C₁₀ olefin composition which can be utilized in the processes described herein can comprise branched C₁₀ olefins; or alternatively, branched C₁₀ alpha olefins. In other aspects, and in addition to the branched C₁₀ olefins (or branched C₁₀ alpha olefins), the C₁₀ olefin composition can further comprise linear C₁₀ olefins (i.e., a mixture comprising branched C₁₀ olefins (or branched C₁₀ alpha olefins) and linear C₁₀ olefins). In an aspect, the linear C₁₀ olefins can be linear alpha olefins. The identity of the branched C₁₀ olefins (or branched C₁₀ alpha olefins), the amount(s) of each branched C₁₀ olefins (or branched C₁₀ alpha olefins), the identity of linear C₁₀ olefins, the amounts of each linear C₁₀ olefins which can be present in the C₁₀ olefin composition are independently described herein and these independent descriptions can be utilized in any combination to further describe the C₁₀ olefins present in the C₁₀ olefin composition utilized for the processes described herein.

In an aspect, the C₁₀ olefin composition which can be utilized in the processes disclosed herein can comprise at least 50 mole %, 60 mole %, 65 mole %, 70 mole %, 75 mole %, 80 mole %, 85 mole %, 90 mole %, or 95 mole % branched C₁₀ olefins (or branched C₁₀ alpha olefins); alternatively or additionally, less than or equal to 99.5 mole %, 99 mole %, 98 mole %, 97 mole %, 95 mole %, 92 mole %, or 90 mole % branched C₁₀ olefins (or branched C₁₀ alpha olefins). Generally, the C₁₀ olefin composition can comprise branched C₁₀ olefins (or branched C₁₀ alpha olefins) ranging from any minimum branched C₁₀ olefins (or branched C₁₀ alpha olefins) content disclosed herein to any maximum branched C₁₀ olefins (or branched C₁₀ alpha olefins) content disclosed herein. For example, in some aspects, the C₁₀ olefin composition can comprise from 50 mole % to 99.5 mole %, from 65 mole % to 99 mole %, from 75 mole % to 99 mole %, from 85 mole % to 97 mole %, from 80 mole % to 95 mole %, from 70 mole % to 95 mole %, or from 75 mole % to 90 mole % branched C₁₀ olefins (or branched C₁₀ alpha olefins). Other ranges for the branched C₁₀ olefins (or branched C₁₀ alpha olefins) within the C₁₀ olefin composition are readily apparent to those skilled in the art with the aid of this disclosure.

In an aspect, the branched C₁₀ olefins (or branched C₁₀ alpha olefins) of the C₁₀ olefin composition can comprise, or can consist essentially of 3-propyl-1-heptene, 4-ethyl-1-octene, 5-methyl-1-nonene, or any combination thereof; alternatively, 3-propyl-1-heptene, 4-ethyl-1-octene, and 5-methyl-1-nonene; or alternatively, 3-propyl-1-heptene, 4-ethyl-1-octene, 5-methyl-1-nonene, and 2-butyl-1-hexene. In an aspect, the branched C₁₀ olefins (or branched C₁₀ alpha olefins) of the C₁₀ olefin composition can comprise i) at least 8 mole %, at least 9 mole %, at least 10 mole %, at least 11 mole %, at least 12 mole %, or at least 13 mole % 3-propyl-1-heptene, ii) at least 6 mole %, at least 7 mole %, at least 8 mole %, at least 9 mole %, at least 10 mole %, or at least 11 mole % 4-ethyl-1-octene, and/or iii) at least 20 mole %, at least 22 mole %, at least 24 mole %, at least 26 mole %, at least 28 mole %, or at least 30 mole % 5-methyl-1-nonene; alternatively, i) at least 8 mole %, at least 9 mole %, at least 10 mole %, at least 11 mole %, at least 12 mole %, or at least 13 mole % 3-propyl-1-heptene, ii) at least 6 mole %, at least 7 mole %, at least 8 mole %, at least 9 mole %, at least 10 mole %, and/or at least 11 mole % 4-ethyl-1-octene, iii) at least 20 mole %, at least 22 mole %, at least 24 mole %, at least 26 mole %, at least 28 mole %, or at least 30 mole % 5-methyl-1-nonene, and iv) at least 3 mole %, at least 4 mole %, at least 5 mole %, at least 6 mole %, at least 7 mole %, or at least 8 mole % 2-butyl-1-hexene. In another aspect, the branched C₁₀ olefins (or branched C₁₀ alpha olefins) of the C₁₀ olefin composition can comprise i) from 8 mole % to 35 mole %, from 10 mole % to 35 mole %, from 11 mole % to 30 mole %, from 12 mole % to 28 mole %, from 13 mole % to 26 mole %, or from 14 mole % to 24 mole % 3-propyl-1-heptene, ii) from 7 mole % to 30 mole %, from 7 mole % to 30 mole %, from 8 mole % to 25 mole %, from 9 mole % to 23 mole %, from 10 mole % to 22 mole %, or from 11 mole % to 21 mole % 4-ethyl-1-octene, and/or iii) from 24 mole % to 65 mole %, from 24 mole % to 65 mole %, from 26 mole % to 60 mole %, from 28 mole % to 55 mole %, from 30 mole % to 50 mole %, or from 32 mole % to 48 mole % 5-methyl-1-nonene; alternatively, i) from 8 mole % to 35 mole %, from 10 mole % to 35 mole %, from 11 mole % to 30 mole %, from 12 mole % to 28 mole %, from 13 mole % to 26 mole %, or from 14 mole % to 24 mole % 3-propyl-1-heptene, ii) from 7 mole % to 30 mole %, from 7 mole % to 30 mole %, from 8 mole % to 25 mole %, from 9 mole % to 23 mole %, from 10 mole % to 22 mole %, or from 11 mole % to 21 mole % 4-ethyl-1-octene, iii) from 24 mole % to 65 mole %, from 24 mole % to 65 mole %, from 26 mole % to 60 mole %, from 28 mole % to 55 mole %, from 30 mole % to 50 mole %, or from 32 mole % to 48 mole % 5-methyl-1-nonene, and/or iv) from 3 mole % to 25 mole %, from 4 mole % to 22 mole %, from 5 mole % to 20 mole %, from 6 mole % to 18 mole %, or from 7 mole % to 16 mole % 2-butyl-1-hexene.

In an aspect, the C₁₀ olefin composition which can be utilized in the processes disclosed herein can comprise a maximum of 50 mole %, 40 mole %, 30 mole %, 25 mole %, 20 mole %, 15 mole % or 10 mole % linear C₁₀ olefins (or linear C₁₀ alpha olefins); alternatively or additionally, the C₁₀ olefin composition can comprise a minimum of 0 mole %, 0.5 mole %, 1 mole %, 1.5 mole %, 2 mole %, or 2.5 mole % linear C₁₀ olefins (or linear C₁₀ alpha olefins). Generally, the C₁₀ olefin composition can comprise linear C₁₀ olefins (or linear C₁₀ alpha olefins) ranging from any minimum linear C₁₀ olefin (or linear C₁₀ alpha olefins) content disclosed herein to any maximum linear C₁₀ olefin (or linear C₁₀ alpha olefins) content disclosed herein. For example, in some non-limiting aspects, the C₁₀ olefin composition can comprise from 0 mole % to 50 mole %, from 0.5 mole % to 40 mole %, from 1 mole % to 30 mole %, from 1.5 mole % to 25 mole %, from 2 mole % to 25 mole %, or from 2.5 mole % to 20 mole % linear C₁₀ olefins (or linear C₁₀ alpha olefins). Other ranges for the linear C₁₀ olefins (or linear C₁₀ alpha olefins) within the C₁₀ olefin composition are readily apparent to those skilled in the art with the aid of this disclosure.

In an aspect, the linear C₁₀ olefins (or linear C₁₀ alpha olefins) present in the C₁₀ olefin composition can comprise (or consist essentially of, or consist of) 1-decene; alternatively, 4- and/or 5-decene; or alternatively, 1-decene, and 4- and/or 5-decene. In some aspects, the C₁₀ olefin composition can comprise a maximum of 40 mole %, 30 mole %, 25 mole %, 20 mole %, 15 mole % or 10 mole % 1-decene; alternatively, or additionally, the C₁₀ olefin composition can comprise a minimum of 0 mole %, 0.5 mole %, 1 mole %, 1.5 mole %, 2 mole %, or 2.5 mole % 1-decene. Generally, the C₁₀ olefin composition can comprise 1-decene ranging from any minimum 1-decene content disclosed herein to any maximum 1-decene content disclosed herein. For example, in some non-limiting aspects, the C₁₀ olefin composition can comprise from 0 mole % to 40 mole %, 0.5 mole % to 30 mole %, 1 mole % to 25 mole %, 1 mole % to 20 mole %, 1 mole % to 15 mole %, 1.5 mole % to 15 mole %, or 1.5 mole to 10 mole % 1-decene. In other aspects, the C₁₀ olefin composition can comprise a maximum of 25 mole %, 22.5 mole %, 20 mole %, 19 mole %, or 18 mole % 4- and/or 5-decene; alternatively or additionally, C₁₀ olefin composition can comprise a minimum of 0 mole %, 1 mole %, 2 mole %, 3 mole %, 4 mole %, or 5 mole % 4- and/or 5-decene. For example, in some non-limiting aspects, the C₁₀ olefin composition can comprise from 0 mole % to 25 mole %, 1 mole % to 20 mole %, 2 mole % to 19 mole %, 3 mole % to 18 mole %, 4 mole % to 17 mole %, 4 mole % to 18 mole %, or 5 mole % to 18 mole % 4- and/or 5-decene. Other ranges for 1-decene, and 4- and/or 5-decene within the C₁₀ olefin composition are readily apparent to those skilled in the art with the aid of this disclosure.

In some aspects, the C₁₀ olefin composition is a C₁₀ olefin composition containing C₁₀ olefins as described herein that is substantially devoid of heteroatomic compounds. Examples of heteroatomic compounds include amines (e.g., pyrroles), peroxides, and alcohols (e.g., ethyl hexanol). “Substantially devoid of heteroatomic compounds” as used herein means a concentration of heteroatomic compounds which is less than 1, 0.1, 0.01, 0.001, or 0.0001 mass % based on a total mass of the C₁₀ olefin composition.

### C₂₀ 2-Substituted Alpha Olefins

In aspects, the process can include contacting a branched C₁₀ olefin composition with a dimerization catalyst or a dimerization catalyst system to form a C₂₀ 2-substituted alpha olefin composition.

The temperature which can be utilized to form the C₂₀ 2-substituted alpha olefin composition can be any temperature capable of forming the C₂₀ 2-substituted alpha olefin composition. In an aspect, the minimum temperature which can be utilized for forming the C₂₀ 2-substituted alpha olefin composition can be −60° C., −30° C., 0° C., 20° C., 50° C., 75° C., or 100° C.; alternatively or additionally, the maximum temperature which can be utilized for forming the C₂₀ 2-substituted alpha olefin composition can be 280° C., 250° C., 230° C., 200° C., 175° C., 150° C., or 125° C. Ranges of temperature which can be utilized which can be utilized for forming the C₂₀ 2-substituted alpha olefin composition can range from any minimum temperature to any maximum temperature described herein for dimerization conditions. In some aspects, suitable ranges for the temperature which can be utilized as dimerization conditions be include, but are not limited to, from −60° C. to 280° C.; alternatively, from −30° C. to 250° C.; alternatively, from 0° C. to 230° C.; alternatively, from 100° C. to 250° C.; alternatively, from 100° C. to 230° C.; alternatively, from 100° C. to 200° C.; alternatively, from 0° C. to 150° C.; alternatively, from 0° C. to 125° C.; or alternatively, from 20° C. to 100° C.

The pressure which can be utilized to form the C₂₀ 2-substituted alpha olefin composition can be any pressure capable of forming the C₂₀ 2-substituted alpha olefin composition. In an aspect, the minimum pressure which can be utilized for forming the C₂₀ 2-substituted alpha olefin composition can be 10 psia (69 kPa), or 14.0 psia (97 kPa), 14.7 psia (101 kPa), or 20 psia (138); alternatively or additionally, the maximum pressure which can be utilized for forming the C₂₀ 2-substituted alpha olefin composition can be 1,000 psia (6.9 MPa), 500 psia (3.4 MPa), 400 psia (2.8 MPa), 300 psia (2 MPa), 200 psia (1.4 MPa), or 100 psia (689 kPa). Ranges of pressure which can be utilized for forming the C₂₀ 2-substituted alpha olefin composition can range from any minimum pressure described herein to any maximum pressure described herein. In some aspects, suitable ranges for the pressure which can be utilized to form the C₂₀ 2-substituted alpha olefin composition can include, but are not limited to, from 10 psia (69 kPa) to 1,000 psia (6.9 MPa), from 10 psia (69 kPa) to 500 psia (3.4 MPa), from 14 psia (97 kPa) to 400 psia (2.8 MPa), from 14 psia (97 kPa) to 300 psia (3.4 MPa), from 14.7 psia (101 kPa) to 200 psia (1.4 MPa), or from 14.7 psia (101 kPa) to 100 psia (689 KPa).

The dimerization reaction can include a reaction time that is any time that can produce the desired quantity of C₂₀ 2-substituted alpha olefin composition; alternatively, any time that can provide a desired dimerization catalyst or catalyst system productivity; alternatively, any time that can provide a desired conversion of a C₁₀ olefin composition disclosed herein (e.g., a conversion of at least 50 wt %; alternatively, at least 60 wt %; alternatively, at least 70 wt %; alternatively, at least 80 wt %). The minimum time (or minimum average time) can be 1 minute, 10 minutes, 30 minutes, 45 minutes, or 1 hour; alternatively or additionally, the maximum time (or average maximum time) can be 48 hours, 36 hours, 24 hours, 12 hours, 6 hours, 4 hours, or 2 hours. Ranges of reaction time which can be utilized for forming the C₂₀ 2-substituted alpha olefin composition can range from any minimum time described herein to any maximum time described herein. In some aspects, suitable ranges for the reaction time which can be utilized as dimerization conditions be include, but are not limited to, from 1 minute to 48 hours, from 10 minutes to 36 hours, from 30 minutes to 24 hours, from 45 minutes to 24 hours, from 1 hour to 12 hours, from 1 hour to 6 hours or from 1 hour to 2 hours.

In aspects, any suitable dimerization catalyst or dimerization catalyst system which can produce the desired the C₂₀ 2-substituted alpha olefins can be used in the process to produce the C₂₀ 2-substituted alpha olefins.

Non-limiting examples of dimerization catalysts and dimerization catalyst systems which can be used can comprise i) an alkylaluminum compound, ii) a zirconium compound, or iii) a metallocene compound, alternatively, i) an alkylaluminum compound, and/or ii) a zirconium compound; alternatively, i) an alkylaluminum compound, or ii) a metallocene compound; alternatively, an alkylaluminum compound, alternatively, a zirconium compound, or alternatively, a metallocene compound.

In an aspect, the C₂₀ 2-substituted alpha olefins can be produced using a catalyst or catalyst system comprising (or consisting essentially of, or consisting of) an alkylaluminum compound. In an aspect, the alkylaluminum compound can comprise, or consist essentially of, a trialkylaluminum compound. In an aspect, the trialkylaluminum compound can comprise, or consist essentially of, singly or in any combination, triethylaluminum, triethylaluminum, tripropylaluminum (e.g., tri-n-propylaluminum and/or tri-2-propylaluminum), tributylaluminum (e.g., tri-n-butylaluminum, tri-2-butylaluminum, and/or tri-t-butyl aluminum), trihexylaluminum, or trioctylaluminum. Other suitable alkylaluminum compounds (and trialkylaluminum compounds) are known to those skilled in the art. Any suitable conditions for dimerizing the alpha olefins with the catalyst or catalyst system comprising (or consisting essentially of, or consisting of) an alkylaluminum compound can be employed.

In an aspect, the C₂₀ 2-substituted alpha olefins can be produced using a dimerization catalyst or dimerization catalyst system comprising a zirconium compound. In an aspect, the catalyst system can comprise a zirconium compound and an alkylaluminum compound; alternatively, can comprise a zirconium compound and an aluminoxane; or alternatively can comprise a zirconium compound, an alkylaluminum compound, and an aluminoxane. Generally, the zirconium compound can be any compound that when combined with the alkylaluminum compound (or aluminoxane, or alkylaluminum compound and aluminoxane) can dimerize an alpha olefin to produce a 2-substituted alpha olefin. In an aspect, the zirconium compound can be a zirconium halide compound; alternatively, a dicyclopentadienyl zirconium halide compound; alternatively, a dicyclopentadienyl zirconium dihalide; or alternatively, dicyclopentadienyl zirconium dichloride. In an aspect, the alkylaluminum compound which can be utilized with the zirconium compound of the dimerization catalyst systems disclosed herein can comprise an alkylaluminum dihalide, an alkylaluminum sesquihalide, an dialkylaluminum halide, a trialkylaluminum compound, or any combination thereof; alternatively, alkylaluminum dihalide; alternatively, an alkylaluminum sesquihalide; alternatively, an dialkylaluminum halide; or alternatively, a trialkylaluminum compound. In an aspect, the aluminoxane which can be utilized with the zirconium compound of the dimerization catalyst systems disclosed herein can comprise (or consist essentially of, or consist of) methylaluminoxane (MAO), ethylaluminoxane, a modified methylaluminoxane (MMAO), n-propylaluminoxane, iso-propyl-aluminoxane, n-butylaluminoxane, sec-butylaluminoxane, iso-butylaluminoxane, t-butylaluminoxane, 1-pentylaluminoxane, 2-pentylaluminoxane, 3-pentyl-aluminoxane, iso-pentyl-aluminoxane, neopentylaluminoxane, or any combination thereof. In one particular aspect, the dimerization catalyst system can comprise (or consist essentially of, or consist of) dicyclopentadienyl zirconium dichloride and any aluminoxane disclosed herein.

In an aspect, the C₂₀ 2-substituted alpha olefins can be produced using a catalyst system comprising a metallocene compound. In an aspect, the catalyst system can comprise a metallocene compound and an aluminoxane, or a metallocene compound, a non-coordinating anion activator, and an alkylaluminum compound; alternatively, a metallocene compound and an aluminoxane; or alternatively, a metallocene compound, a non-coordinating anion activator, and an alkylaluminum compound. Generally, the metallocene compound can be any metallocene compound that, when utilized in the presence of the other catalyst system components, can dimerize the alpha olefins to 2-substituted alpha olefins. Suitable metallocenes, aluminoxane and/or alkylaluminum compounds, and non-coordinating anion activators which can be utilized the dimerization catalyst system comprising a metallocene are disclosed in U.S. Pat. Nos. 6,548,723, 7,989,670, 8,207,390, and 8,536,391, among other documents.

A product, or a portion of the C₁₀ dimerization product, of the process(es) described herein is a composition comprising C₂₀ 2-substituted alpha olefins (also referred to as a C₂₀ 2-substituted alpha olefin composition). In aspects, the C₂₀ 2-substituted alpha olefin composition, can comprise at least 50, 60, 70, 75, 80, 85, 90, or 95 mole % C₂₀ 2-substituted alpha olefins.

Description of the C₂₀ 2-substituted alpha olefins is divided into three groups; a first group of C₂₀ 2-substituted alpha olefins, a second group of C₂₀ 2-substituted alpha olefins, and a third group of C₂₀ 2-substituted alpha olefins. The description of the C₂₀ 2-substituted alpha olefins of the composition comprising C₂₀ 2-substituted alpha olefins can include one or more of the C₂₀ 2-substituted alpha olefin(s) selected from the first group; alternatively, one or more of branched C₂₀ 2-substituted alpha olefins selected from the first group and one or more of the C₂₀ 2-substituted alpha olefins selected from the second group; or alternatively, one or more of the branched C₂₀ 2-substituted alpha olefin(s) selected from the first group, one or more of the C₂₀ 2-substituted alpha olefin(s) selected from the second group, and one or more of the C₂₀ 2-substituted alpha olefin(s) selected from the third group.

In aspects, the C₂₀ 2-substituted alpha olefins of the first group can include 2-(3-methylheptyl)-7-methyl-1-undecene, 2-(4-octyl)-7-methyl-1-undecene, 2-(3-methylheptyl)-5-propyl-1-nonene, 2-(2-ethylhexyl)-7-methyl-1-undecene, 2-(3-methylheptyl)-6-ethyl-1-decene, or any combination thereof. In one aspect, the C₂₀ 2-substituted alpha olefins can comprise, only one, only two, only three, or only four, of 2-(3-methylheptyl)-7-methyl-1-undecene, 2-(4-octyl)-7-methyl-1-undecene, 2-(3-methylheptyl)-5-propyl-1-nonene, 2-(2-ethylhexyl)-7-methyl-1-undecene, and 2-(3-methylheptyl)-6-ethyl-1-decene. In another aspect, the C₂₀ 2-substituted alpha olefins can comprise 2-(3-methylheptyl)-7-methyl-1-undecene, 2-(4-octyl)-7-methyl-1-undecene, 2-(3-methylheptyl)-5-propyl-1-nonene, 2-(2-ethylhexyl)-7-methyl-1-undecene, and 2-(3-methylheptyl)-6-ethyl-1-decene.

In an aspect, the C₂₀ 2-substituted alpha olefins can include any minimum amount of 2-(3-methylheptyl)-7-methyl-1-undecene, 2-(4-octyl)-7-methyl-1-undecene, 2-(3-methylheptyl)-5-propyl-1-nonene, 2-(2-ethylhexyl)-7-methyl-1-undecene, and/or 2-(3-methylheptyl)-6-ethyl-1-decene disclosed herein. In an aspect, 2-(3-methylheptyl)-7-methyl-1-undecene, when present, can comprise at least 10, 11, 12, 13, 14, 15, 16, 17, 18, 19, or 20 mole % of the C₂₀ 2-substituted alpha olefins; alternatively or additionally, a maximum of (less than or equal to) 50, 45, 40, 38, 36, 32, or 30 mole % of the C₂₀ 2-substituted alpha olefins. In another aspect, 2-(3-methylheptyl)-7-methyl-1-undecene, when present, can comprise from any minimum mole % of the C₂₀ 2-substituted alpha olefins described herein to any maximum mole % of the C₂₀ 2-substituted alpha olefins described herein; for example from 10 mole % to 50 mole %, from 12 to 45 mole %, from 15 to 40 mole %, from 18 to 38 mole %, from 17 to 36 mole %, from 18 to 34 mole %, from 19 to 32 mole %, or from 20 to 30 mole % 2-(3-methylheptyl)-7-methyl-1-undecene. In an aspect, 2-(4-octyl)-7-methyl-1-undecene, when present, can comprise at least 3, 4, 5, 6, or 7 mole % of the C₂₀ 2-substituted alpha olefins; alternatively or additionally, a maximum of (less than or equal to) 25, 21, 19, 17, 15 mole % of the C₂₀ 2-substituted alpha olefins. In another aspect, 2-(4-octyl)-7-methyl-1-undecene, when present, can comprise from any minimum mole % of the C₂₀ 2-substituted alpha olefins described herein to any maximum mole % of the C₂₀ 2-substituted alpha olefins described herein; for example from 3 mole % to 25 mole %, from 4 to 21 mole %, from 5 to 19 mole %, from 6 to 17 mole %, or from 7 to 15 mole % 2-(4-octyl)-7-methyl-1-undecene. In an aspect, 2-(3-methylheptyl)-5-propyl-1-nonene, when present, can comprise at least 3, 4, 5, 6, or 7 mole % of the C₂₀ 2-substituted alpha olefins; alternatively or additionally, a maximum of (less than or equal to) 25, 21, 19, 17, 15 mole % of the C₂₀ 2-substituted alpha olefins. In another aspect, 2-(3-methylheptyl)-5-propyl-1-nonene, when present, can comprise from any minimum mole % of the C₂₀ 2-substituted alpha olefins described herein to any maximum mole % of the C₂₀ 2-substituted alpha olefins described herein; for example from 3 mole % to 25 mole %, from 4 to 21 mole %, from 5 to 19 mole %, from 6 to 17 mole %, or from 7 to 15 mole % 2-(3-methylheptyl)-5-propyl-1-nonene; In an aspect, 2-(2-ethylhexyl)-7-methyl-1-undecene, when present, can comprise at least 3, 4, 5, 6, or 7 mole % of the C₂₀ 2-substituted alpha olefins; alternatively or additionally, a maximum of (less than or equal to) 25, 21, 19, 17, 15 mole % of the C₂₀ 2-substituted alpha olefins. In another aspect, 2-(2-ethylhexyl)-7-methyl-1-undecene, when present, can comprise from any minimum mole % of the C₂₀ 2-substituted alpha olefins described herein to any maximum mole % of the C₂₀ 2-substituted alpha olefins described herein; for example from 3 mole % to 25 mole %, from 4 to 21 mole %, from 5 to 19 mole %, from 6 to 17 mole %, or from 7 to 15 mole % 2-(2-ethylhexyl)-7-methyl-1-undecene. In an aspect, 2-(3-methylheptyl)-6-ethyl-1-decene, when present, can comprise at least 5, 6, or 7 mole % of the C₂₀ 2-substituted alpha olefins; alternatively or additionally, a maximum of (less than or equal to) 25, 21, 19, 17, 15 mole % of the C₂₀ 2-substituted alpha olefins. In another aspect, 2-(3-methylheptyl)-6-ethyl-1-decene, when present, can comprise from any minimum mole % of the C₂₀ 2-substituted alpha olefins described herein to any maximum mole % of the C₂₀ 2-substituted alpha olefins described herein; for example from 3 mole % to 25 mole %, from 4 to 21 mole %, from 5 to 19 mole %, from 6 to 17 mole %, or from 7 to 15 mole % 2-(3-methylheptyl)-6-ethyl-1-decene.

In aspects, the C₂₀ 2-substituted alpha olefins can additionally include C₂₀ 2-substituted alpha olefins selected from the second group of C₂₀ 2-substituted alpha olefins: i.e., 2-(4-octyl)-5-propyl-1-nonene, 2-(4-octyl)-6-ethyl-1-decene, 2-(2-ethylhexyl)-5-propyl-1-nonene, 2-(2-ethylhexyl)-6-ethyl-1-decene, or any combination thereof. In one aspect, the C₂₀ 2-substituted alpha olefins can further comprise only one, only two, or only three of 2-(4-octyl)-5-propyl-1-nonene, 2-(4-octyl)-6-ethyl-1-decene, 2-(2-ethylhexyl)-5-propyl-1-nonene, and 2-(2-ethylhexyl)-6-ethyl-1-decene. In another aspect, the C₂₀ 2-substituted alpha olefins can further comprise 2-(4-octyl)-5-propyl-1-nonene, 2-(4-octyl)-6-ethyl-1-decene, 2-(2-ethylhexyl)-5-propyl-1-nonene, and 2-(2-ethylhexyl)-6-ethyl-1-decene.

In an aspect, the C₂₀ 2-substituted alpha olefins can further comprise any amount of 2-(4-octyl)-5-propyl-1-nonene disclosed herein, 2-(4-octyl)-6-ethyl-1-decene disclosed herein, 2-(2-ethylhexyl)-5-propyl-1-nonene disclosed herein, and/or 2-(2-ethylhexyl)-6-ethyl-1-decene disclosed herein. In an aspect, the 2-(4-octyl)-5-propyl-1-nonene, when present, can comprise at least 3, 3.5, or 4 mole % of the C₂₀ 2-substituted alpha olefins; alternatively or additionally, a maximum of (less than or equal to) 9, 8.5, 8, 7.5, or 7 mole % of the C₂₀ 2-substituted alpha olefins. In another aspect, 2-(4-octyl)-5-propyl-1-nonene, when present, can comprise from any minimum mole % of the C₂₀ 2-substituted alpha olefins described herein to any maximum mole % of the C₂₀ 2-substituted alpha olefins described herein; for example from 3 mole % to 9 mole %, from 3 mole % to 8.5 mole %, from 3.5 mole % to 8 mole %, from 4 mole % to 7.5 mole %, or from 4 mole % to 7 mole % 2-(4-octyl)-5-propyl-1-nonene. In an aspect, the 2-(4-octyl)-6-ethyl-1-decene, when present, can comprise at least 3, 3.5, or 4 mole % of the C₂₀ 2-substituted alpha olefins; alternatively or additionally, a maximum of (less than or equal to) 9, 8, 7, 6.5, or 6 mole % of the C₂₀ 2-substituted alpha olefins. In another aspect, 2-(4-octyl)-6-ethyl-1-decene, when present, can comprise from any minimum mole % of the C₂₀ 2-substituted alpha olefins described herein to any maximum mole % of the C₂₀ 2-substituted alpha olefins described herein; for example from 3 mole % to 9 mole %, from 3 mole % to 8 mole %, from 3.5 mole % to 7 mole %, from 4 mole % to 6.5 mole %, or from 4 mole % to 6 mole % 2-(4-octyl)-6-ethyl-1-decene. In an aspect, the 2-(2-ethylhexyl)-5-propyl-1-nonene, when present, can comprise at least 3, 3.5, or 4 mole % of the C₂₀ 2-substituted alpha olefins; alternatively or additionally, a maximum of (less than or equal to) 9, 8, 7, 6.5, or 6 mole % of the C₂₀ 2-substituted alpha olefins. In another aspect, 2-(2-ethylhexyl)-5-propyl-1-nonene, when present, can comprise from any minimum mole % of the C₂₀ 2-substituted alpha olefins described herein to any maximum mole % of the C₂₀ 2-substituted alpha olefins described herein; for example from 3 mole % to 9 mole %, from 3 mole % to 8 mole %, from 3.5 mole % to 7 mole %, from 4 mole % to 6.5 mole %, or from 4 mole % to 6 mole % 2-(2-ethylhexyl)-5-propyl-1-nonene. In an aspect, the 2-(2-ethylhexyl)-6-ethyl-1-decene, when present, can comprise at least 2, 2.5, or 3 mole % of the C₂₀ 2-substituted alpha olefins; alternatively or additionally, a maximum of (less than or equal to) 8, 7, 6.5, or 6 mole % of the C₂₀ 2-substituted alpha olefins. In another aspect, 2-(2-ethylhexyl)-6-ethyl-1-decene, when present, can comprise from any minimum mole % of the C₂₀ 2-substituted alpha olefins described herein to any maximum mole % of the C₂₀ 2-substituted alpha olefins described herein; for example from 2 mole % to 8 mole %, from 2 mole % to 7 mole %, from 2.5 mole % to 6.5 mole %, or from 3 mole % to 6 mole % 2-(2-ethylhexyl)-6-ethyl-1-decene.

In aspects, the C₂₀ 2-substituted alpha olefins can additionally include C₂₀ 2-substituted alpha olefins selected from the third group of C₂₀ 2-substituted alpha olefins: i.e., 2-(3-methylheptyl)-1-dodecene, 2-octyl-7-methyl-1-undecene, 2-(4-octyl)-1-dodecene, 2-(3-propylheptyl)-1-decene, 2-(2-ethylhexyl)-1-dodecene, 2-octyl-6-ethyl-1-decene, 2-octyl-1-dodecene, or any combination thereof. In one aspect, the C₂₀ 2-substituted alpha olefins can further comprise only one, only two, only three, only four, only five, or only six of 2-(3-methylheptyl)-1-dodecene, 2-octyl-7-methyl-1-undecene, 2-(4-octyl)-1-dodecene, 2-(3-propylheptyl)-1-decene, 2-(2-ethylhexyl)-1-dodecene, 2-octyl-6-ethyl-1-decene, and 2-octyl-1-dodecene. In another aspect, the C₂₀ 2-substituted alpha olefins can further comprise 2-(3-methylheptyl)-1-dodecene, 2-octyl-7-methyl-1-undecene, 2-(4-octyl)-1-dodecene, 2-(3-propylheptyl)-1-decene, 2-(2-ethylhexyl)-1-dodecene, 2-octyl-6-ethyl-1-decene, and 2-octyl-1-dodecene.

In an aspect, the C₂₀ 2-substituted alpha olefins can further comprise any amount of 2-(3-methylheptyl)-1-dodecene disclosed herein, 2-octyl-7-methyl-1-undecene disclosed herein, 2-(4-octyl)-1-dodecene disclosed herein, 2-(3-propylheptyl)-1-decene disclosed herein, 2-(2-ethylhexyl)-1-dodecene disclosed herein, 2-octyl-6-ethyl-1-decene disclosed herein, and/or 2-octyl-1-dodecene disclosed herein. In an aspect, the 2-(3-methylheptyl)-1-dodecene, when present, can comprise at least 0.5, 1, 1.5, or 2 mole % of the C₂₀ 2-substituted alpha olefins; alternatively, or additionally, a maximum of (less than or equal to) 8, 7, 6, 5, or 4 mole % of the C₂₀ 2-substituted alpha olefins. In another aspect, 2-(3-methylheptyl)-1-dodecene, when present, can comprise from any minimum mole % of the C₂₀ 2-substituted alpha olefins described herein to any maximum mole % of the C₂₀ 2-substituted alpha olefins described herein; for example from 0.5 mole % to 8 mole %, from 0.5 mole % to 7 mole %, from 1 mole % to 6 mole %, from 1.5 mole % to 5 mole %, or from 2 mole % to 4 mole % 2-(3-methylheptyl)-1-dodecene. In an aspect, the 2-octyl-7-methyl-1-undecene, when present, can comprise at least 0.5, 1, 1.5, or 2 mole % of the C₂₀ 2-substituted alpha olefins; alternatively, or additionally, a maximum of (less than or equal to) 8, 7, 6, 5, or 4 mole % of the C₂₀ 2-substituted alpha olefins. In another aspect, 2-octyl-7-methyl-1-undecene, when present, can comprise from any minimum mole % of the C₂₀ 2-substituted alpha olefins described herein to any maximum mole % of the C₂₀ 2-substituted alpha olefins described herein; for example from 0.5 mole % to 8 mole %, from 0.5 mole % to 7 mole %, from 1 mole % to 6 mole %, from 1.5 mole % to 5 mole %, or from 2 mole % to 4 mole % 2-octyl-7-methyl-1-undecene. In an aspect, the 2-(4-octyl)-1-dodecene, when present, can comprise at least 0.5, 0.75, or 1 mole % of the C₂₀ 2-substituted alpha olefins; alternatively, or additionally, a maximum of (less than or equal to) 6, 5, 4, 3, or 2 mole % of the C₂₀ 2-substituted alpha olefins. In another aspect, 2-(4-octyl)-1-dodecene, when present, can comprise from any minimum mole % of the C₂₀ 2-substituted alpha olefins described herein to any maximum mole % of the C₂₀ 2-substituted alpha olefins described herein; for example from 0.5 mole % to 6 mole %, from 0.5 mole % to 5 mole %, from 0.75 mole % to 4 mole %, from 0.75 mole % to 3 mole %, or from 1 mole % to 2 mole % 2-(4-octyl)-1-dodecene. In an aspect, the 2-(3-propylheptyl)-1-decene, when present, can comprise at least 0.5, 0.75, or 1 mole % of the C₂₀ 2-substituted alpha olefins; alternatively, or additionally, a maximum of (less than or equal to) 6, 5, 4, 3, or 2 mole % of the C₂₀ 2-substituted alpha olefins. In another aspect, 2-(3-propylheptyl)-1-decene, when present, can comprise from any minimum mole % of the C₂₀ 2-substituted alpha olefins described herein to any maximum mole % of the C₂₀ 2-substituted alpha olefins described herein; for example from 0.5 mole % to 6 mole %, from 0.5 mole % to 5 mole %, from 0.75 mole % to 4 mole %, from 0.75 mole % to 3 mole %, or from 1 mole % to 2 mole % 2-(3-propylheptyl)-1-decene. In an aspect, the 2-(2-ethylhexyl)-1-dodecene, when present, can comprise at least 0.5, 0.75, or 1 mole % of the C₂₀ 2-substituted alpha olefins; alternatively, or additionally, a maximum of (less than or equal to) 6, 5, 4, 3, or 2 mole % of the C₂₀ 2-substituted alpha olefins. In another aspect, 2-(2-ethylhexyl)-1-dodecene, when present, can comprise from any minimum mole % of the C₂₀ 2-substituted alpha olefins described herein to any maximum mole % of the C₂₀ 2-substituted alpha olefins described herein; for example from 0.5 mole % to 6 mole %, from 0.5 mole % to 5 mole %, from 0.75 mole % to 4 mole %, from 0.75 mole % to 3 mole %, or from 1 mole % to 2 mole % 2-(2-ethylhexyl)-1-dodecene. In an aspect, the 2-octyl-6-ethyl-1-decene, when present, can comprise at least 0.5, 0.75, or 1 mole % of the C₂₀ 2-substituted alpha olefins; alternatively, or additionally, a maximum of (less than or equal to) 6, 5, 4, 3, or 2 mole % of the C₂₀ 2-substituted alpha olefins. In another aspect, 2-octyl-6-ethyl-1-decene, when present, can comprise from any minimum mole % of the C₂₀ 2-substituted alpha olefins described herein to any maximum mole % of the C₂₀ 2-substituted alpha olefins described herein; for example from 0.5 mole % to 6 mole %, from 0.5 mole % to 5 mole %, from 0.75 mole % to 4 mole %, from 0.75 mole % to 3 mole %, or from 1 mole % to 2 mole % 2-octyl-6-ethyl-1-decene. In an aspect, the 2-octyl-1-dodecene, when present, can comprise at least 0.1, 0.15, or 0.2 mole % of the C₂₀ 2-substituted alpha olefins; alternatively, or additionally, a maximum of (less than or equal to) 5, 4, 3, 2, or 1 mole % of the C₂₀ 2-substituted alpha olefins. In another aspect, 2-octyl-1-dodecene, when present, can comprise from any minimum mole % of the C₂₀ 2-substituted alpha olefins described herein to any maximum mole % of the C₂₀ 2-substituted alpha olefins described herein; for example from 0.1 mole % to 5 mole %, from 0.1 mole % to 4 mole %, from 0.15 mole % to 3 mole %, from 0.15 mole % to 2 mole %, or from 0.2 mole % to 1 mole % 2-octyl-1-dodecene.

### C₂₀ Trisubstituted Alpha Olefins

In aspects, the process can include contacting the C₂₀ 2-substituted alpha olefin composition with an acidic catalyst to form a C₂₀ trisubstituted olefin composition comprising a C₂₀ trisubstituted olefin. In an aspect, the C₂₀ trisubstituted olefin composition which can be produced according to the processes described herein can comprise one or more C₂₀ trisubstituted olefins formed by contacting the C₂₀ 2-substituted alpha olefin composition with an acidic catalyst.

In aspects, contacting the C₂₀ 2-substituted alpha olefin composition with an acidic catalyst can be performed in any type of reactor which can produce isomers in the C₂₀ trisubstituted olefin composition. In an aspect, contacting the C₂₀ 2-substituted alpha olefin composition with an acidic catalyst can be performed in a batch process, a continuous process; or any combination thereof, alternatively a batch process; or alternatively a continuous process. In some aspects, contacting the C₂₀ 2-substituted alpha olefin composition with an acidic catalyst can be performed in a slurry reactor, a continuous stirred tank reactor, a fixed bed reactor or any combination thereof; alternatively, a slurry reactor; alternatively, a continuous stirred tank reactor; or alternatively, a fixed bed reactor.

In aspects, contacting the C₂₀ 2-substituted alpha olefin composition with an acidic catalyst can be performed at a temperature in a range of from 50° C. to 70° C. It has been found that performing the contacting step at a temperature in this range minimizes dimerization of the C₂₀ 2-substituted alpha olefins in the C₂₀ 2-substituted alpha olefin composition, while allowing for isomerization of C₂₀ trisubstituted olefins to occur to form the C₂₀ trisubstituted olefin composition comprising C₂₀ trisubstituted olefins.

In aspects, contacting the C₂₀ 2-substituted alpha olefin composition with an acidic catalyst can be performed at any pressure capable of forming the C₂₀ trisubstituted olefin composition. In an aspect, the minimum pressure which can be utilized for forming the C₂₀ trisubstituted olefin composition can be 10 psia (69 kPa), or 14.0 psia (97 kPa), 14.7 psia (101 kPa), or 20 psia (138); alternatively or additionally, the maximum pressure which can be utilized for forming the C₂₀ trisubstituted olefin composition can be 1,000 psia (6.9 MPa), 500 psia (3.4 MPa), 400 psia (2.8 MPa), 300 psia (2 MPa), 200 psia (1.4 MPa), or 100 psia (689 kPa). Ranges of pressure which can be utilized for forming the C₂₀ trisubstituted olefin composition can range from any minimum pressure described herein to any maximum pressure described herein. In some aspects, suitable ranges for the pressure which can be utilized to form the C₂₀ trisubstituted olefin composition can include, but are not limited to, from 10 psia (69 kPa) to 1,000 psia (6.9 MPa), from 10 psia (69 kPa) to 500 psia (3.4 MPa), from 14 psia (97 kPa) to 400 psia (2.8 MPa), from 14 psia (97 kPa) to 300 psia (3.4 MPa), from 14.7 psia (101 kPa) to 200 psia (1.4 MPa), or from 14.7 psia (101 kPa) to 100 psia (689 KPa).

In aspects, contacting the C₂₀ 2-substituted alpha olefin composition with an acidic catalyst can be performed at a reaction time that is any time that can produce the desired quantity of C₂₀ trisubstituted olefin composition; alternatively, any time that can provide a desired acidic catalyst or catalyst system productivity; alternatively, any time that can provide a desired conversion of a C₂₀ 2-substituted alpha olefin composition disclosed herein (e.g., a conversion of at least 50 wt %; alternatively, at least 60 wt %; alternatively, at least 70 wt %; alternatively, at least 80 wt %). The minimum time (or minimum average time) can be 1 minute, 10 minutes, 30 minutes, 45 minutes, or 1 hour; alternatively or additionally, the maximum time (or average maximum time) can be 48 hours, 36 hours, 24 hours, 12 hours, 6 hours, 4 hours, or 2 hours. Ranges of reaction time which can be utilized for forming the C₂₀ trisubstituted olefin composition can range from any minimum time described herein to any maximum time described herein. In some aspects, suitable ranges for the reaction time include, but are not limited to, from 1 minute to 48 hours, from 10 minutes to 36 hours, from 30 minutes to 24 hours, from 45 minutes to 24 hours, from 1 hour to 12 hours, from 1 hour to 6 hours or from 1 hour to 2 hours.

### Acidic Catalyst

In aspects, any suitable acidic catalyst which can produce the desired the C₂₀ trisubstituted olefins can be used in the process to produce the C₂₀ trisubstituted olefins.

Non-limiting examples of acidic catalyst which can be used can include an acidic ion exchange resin catalyst, an acidic clay catalyst, an acidic zeolite catalyst, an acidic alumina catalyst, an acidic silicate catalyst, or combinations thereof.

### Acidic Ion Exchange Resin

In aspects, the acidic catalyst can comprise (or consist essentially of, or consist of) an acidic ion exchange resin. In embodiments, the acidic ion exchange resin can comprise (or consist essentially of, or consist of) a styrene-divinylbenzene polymer resin, a functionalized styrene-divinylbenzene polymer resin, a functionalized polymer resin comprising units derived from styrene and units derived from divinyl benzene, a 4-vinylpyridine divinylbenzene polymer resin, an ionomer resin, a tetrafluoroethylene polymer resin modified with perfluorovinyl ether groups terminated with sulfonate groups, or any combination thereof; or alternatively, a styrene-divinylbenzene polymer resin, a functionalized styrene-divinylbenzene polymer resin, a functionalized polymer resin comprising units derived from styrene and units derived from divinyl benzene, or any combination thereof. In yet other embodiment, the acidic catalyst can comprise (or consist essentially of, or consist of) a styrene-divinylbenzene polymer resin; alternatively, a functionalized styrene-divinylbenzene polymer resin; alternatively, a functionalized polymer resin comprising units derived from styrene and units derived from divinyl benzene; alternatively, a 4-vinylpyridine divinylbenzene polymer resin; alternatively, an ionomer resin; or alternatively, a tetrafluoroethylene polymer resin modified with perfluorovinyl ether groups terminated with sulfonate groups.

The acidic ion exchange resin can be embodied as a commercially available acidic resin such as an AMBERLYST® resin, a NAFION® resin, or any combination thereof. Various grades of the AMBERLYST® resin and/or the NAFION® resin can be used as the acidic catalyst. While not limited thereto, the acidic catalyst can comprise (or consist essentially of, or consist of) AMBERLYST® 15 resin, AMBERLYST® 31 resin, AMBERLYST® 35 resin, AMBERLYST® 36 resin, AMBERLYST® DT resin, or any combination thereof; alternatively, AMBERLYST® 15 resin; alternatively, AMBERLYST® 31 resin; alternatively, AMBERLYST® 35 resin; alternatively, AMBERLYST® 36 resin; or alternatively, AMBERLYST® DT resin.

The acidic ion exchange resin can be modified or functionalized with an organic acid and/or an inorganic acid; alternatively, an organic acid; or alternatively, an inorganic acid. In some aspects, the acidic ion exchange resin can be modified with a carboxylic acid, a sulfonic acid, or any combination thereof; alternatively, a carboxylic acid; or alternatively, a sulfonic acid. In aspects, the carboxylic acid can be a C₁ to C₂₀ carboxylic acid; alternatively, a C₁ to C₁₅ carboxylic acid; or alternatively, a C₁ to C₁₀ carboxylic acid. In aspects, the sulfonic acid can be a C₁ to C₂₀ sulfonic acid; alternatively, a C₁ to C₁₅ sulfonic acid; or alternatively, a C₁ to C₁₀ sulfonic acid. In a non-limiting embodiment, the acid which can be utilized to modify the acidic ion exchange resin can comprise, consist essentially of, or consist of, benzoic acid, formic acid, acetic acid, propionic acid, butyric acid, oxalic acid, trifluoroacetic acid, trichloroacetic acid, sulfamic acid, benzene sulfonic acid, toluene sulfonic acid (ortho, meta, and/or para), dodecylbenzene sulfonic acid, naphthalene sulfonic acid, dinonylnaphthalene disulfonic acid, methane sulfonic acid, or any combination thereof; alternatively, benzoic acid, formic acid, acetic acid, propionic acid, butyric acid, oxalic acid, trifluoroacetic acid, trichloroacetic acid, or any combination thereof; or alternatively, benzene sulfonic acid, toluene sulfonic acid (ortho, meta, and/or para), dodecylbenzene sulfonic acid, naphthalene sulfonic acid, dinonylnaphthalene disulfonic acid, methane sulfonic acid, or any combination thereof. In a non-limiting embodiment, the acidic ion exchange resin can be modified or functionalized with an acid comprising, consisting essentially of, or consisting of, benzoic acid; alternatively, formic acid; alternatively, acetic acid; alternatively, propionic acid; alternatively, butyric acid; alternatively, oxalic acid; alternatively, trifluoroacetic acid; alternatively, trichloroacetic acid; alternatively, sulfamic acid; alternatively, benzene sulfonic acid; alternatively, toluene sulfonic acid; alternatively, dodecylbenzene sulfonic acid; alternatively, naphthalene sulfonic acid; alternatively, dinonylnaphthalene disulfonic acid; or alternatively, methane sulfonic acid.

### Acidic Clay

The acidic clay can be any clay material that can catalyze the reaction of C₂₀ 2-substituted alpha olefins which can produce the desired the C₂₀ trisubstituted olefins. In aspects, the acidic clay can comprise, can consist essentially of, or can be, an acidic form of a clay selected from kaolinite, halloysite, vermiculite, chlorite, attapulgite, smectite, montmorillonite, illite, saconite, sepiolite, palygorskite, bentonite, or any combination thereof. The acidic form of any one or combination of the clays can be obtained by washing the clay(s) with an acid, such as HCl, HBr, H₂SO₄, HNO₃, HPO₄, or combinations thereof. Commercially available acidic clays which can be utilized as a catalyst can include those sold by Engineered Clays Corporation, a U.S. Silica company.

### Acidic Zeolite

The acidic zeolite can be any zeolite that can catalyze the reaction of C₂₀ 2-substituted alpha olefins which can produce the desired the C₂₀ trisubstituted olefins. In aspects, the acidic zeolite can comprise, can consist essentially of, or can be, an acidic form of a zeolite, such as zeolite L, zeolite X, zeolite Y, zeolite omega, beta, mordenite, or combinations thereof. The acidic form of any one or combination of the zeolites can be obtained by washing the zeolite(s) with an acid, such as hydrochloric acid, hydrobromic acid, sulfuric acid, nitric acid, phosphoric acid, perchloric acid, sulfamic acid, or combinations thereof. Non-limiting examples of an acidic zeolite include LZ-Y52, LZ-Y62, LZ-Y72, LZ-Y82, and 13-X as described in U.S. Pat. No. 4,697,040.

### Acidic Alumina

The acidic alumina can be any alumina that can catalyze the reaction of C₂₀ 2-substituted alpha olefins which can produce the desired the C₂₀ trisubstituted olefins. In aspects, the acidic alumina can comprise can consist essentially of, or can be, an acidic form of alumina. The acidic form of alumina can be obtained by washing the alumina with an acid, such as hydrochloric acid, hydrobromic acid, sulfuric acid, nitric acid, phosphoric acid, perchloric acid, sulfamic acid, or combinations thereof.

### Acidic Silicate

The acidic silicate can be any silicate that can catalyze the reaction of C₂₀ 2-substituted alpha olefins which can produce the desired the C₂₀ trisubstituted olefins. In aspects, the acidic silicate can comprise, can consist essentially of, or can be, an acidic form of a silicate compound. The acidic form of the silicate can be obtained by washing the silicate with an acid, such as hydrochloric acid, hydrobromic acid, sulfuric acid, nitric acid, phosphoric acid, perchloric acid, sulfamic acid, or combinations thereof.

In aspects, the acidic catalyst can be contained in a catalyst bed in a reactor or vessel that is used to perform the contacting step to form the C₂₀ trisubstituted product via reactions. The catalyst bed can be in a fixed catalyst bed configuration, containing the solid particles of the acidic catalyst. The liquid phase reactants can pass through the catalyst bed, contacting the surface of the catalyst particles as the liquid molecules pass through the catalyst bed. Alternatively, the catalyst bed can be a fluidized bed that contains the solid particles in the reactor or vessel, fluidized by the flow of the reaction medium in the reactor or vessel. In these aspects, the acidic catalyst can be contained in the reactor or vessel by screens that allow the liquid reaction medium to pass out of the reactor or vessel while containing the acidic catalyst particles in the reactor or vessel.

### C₂₀ Trisubstituted Olefin Composition

Specific C₂₀ trisubstituted olefins are identified herein. The naming of the specific C₂₀ trisubstituted olefins is exemplified with 5-methyl-8-methyl-13-methyl-heptadec-7-ene. The structure for 5-methyl-8-methyl-13-methyl-heptadec-7-ene is shown below:

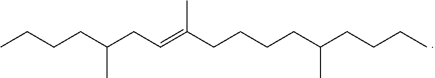 [SMILES: CCCCC(C)C/C=C(\C)CCCCC(C)CCCC ; "."]

The longest linear carbon chain is 17 carbons. Positions on the carbon chain are counted from left to right. The carbon-carbon double bond is between the carbon in the 7 position and the carbon in the 8 position of the linear carbon chain. The first alkyl group is a methyl group on the carbon in the 5 position of the linear carbon chain, the second alkyl group is a methyl group on the carbon in the 8 position of the linear carbon chain, and the third alkyl group is on the carbon in the 13 position of the linear carbon chain.

In another example, the structure for 5-methyl-8-methyl-13-methyl-heptadec-8-ene is shown below:

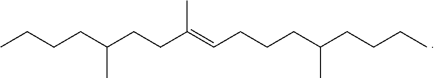 [SMILES: CCCCC(C)CCC/C=C(\C)CCC(C)CCCC ; "."]

The longest linear carbon chain is 17 carbons. Positions on the carbon chain are again counted from left to right. The carbon-carbon double bond is between the carbon in the 8 position and the carbon in the 9 position of the linear carbon chain. The first alkyl group is a methyl group on the carbon in the 5 position of the linear carbon chain, the second alkyl group is a methyl group on the carbon in the 8 position of the linear carbon chain, and the third alkyl group is on the carbon in the 13 position of the linear carbon chain.

In aspects, the C₂₀ trisubstituted olefin composition can contain C₂₀ trisubstituted olefins and at least some of their isomers.

Description of the C₂₀ trisubstituted olefins is divided into three groups; a first group of C₂₀ trisubstituted olefins, a second group of C₂₀ trisubstituted olefin, and a third group of C₂₀ trisubstituted olefins. The C₂₀ trisubstituted olefin composition can include one or more of the C₂₀ trisubstituted olefin(s) selected from the first group; alternatively, one or more of C₂₀ trisubstituted olefin(s) selected from the first group and one or more of the C₂₀ trisubstituted olefin(s) selected from the second group; or alternatively, one or more of the branched C₂₀ trisubstituted olefin(s) n selected from the first group, one or more of the C₂₀ trisubstituted olefin(s) selected from the second group, and one or more of the C₂₀ trisubstituted olefin(s) selected from the third group.

In aspects, the C₂₀ trisubstituted olefin composition can contain a first group of C₂₀ trisubstituted olefins having a linear carbon chain having 13 to 17 carbon atoms, wherein the linear carbon chain has a first alkyl group on a carbon in a 4 or 5 position of the linear carbon chain, wherein the first alkyl group is a methyl group, an ethyl group, a propyl group, or a butyl group, wherein the linear carbon chain has a second alkyl group on a carbon in a 6, 7, or 8 position of the linear carbon chain, wherein the second alkyl group is a methyl group, wherein the linear carbon chain has a third alkyl group on a carbon in a 9, 10, 11, 12, or 13 position of the linear carbon chain, wherein the third alkyl group is a methyl group, an ethyl group, or a propyl group, and wherein a carbon-carbon double bond is on a carbon in the 5, 6, 7, or 8 position of the linear carbon chain.

In some aspects, the first group can contain 5-propyl-6-methyl-9-propyl-tridec-5-ene, 5-propyl-6-methyl-9-propyl-tridec-6-ene, 4-butyl-7-methyl-10-methyl-tetradec-6-ene, 4-butyl-7-methyl-10-methyl-tetradec-7-ene, 5-propyl-6-methyl-10-ethyl-tetradec-5-ene, 5-propyl-6-methyl-10-ethyl-tetradec-6-ene, 5-ethyl-7-methyl-10-propyl-tetradec-6-ene, 5-ethyl-7-methyl-10-propyl-tetradec-7-ene, 5-ethyl-7-methyl-11-ethyl-pentadec-6-ene, 5-ethyl-7-methyl-11-ethyl-pentadec-7-ene, 5-propyl-6-methyl-11-methyl-pentadec-5-ene, 5-propyl-6-methyl-11-methyl-pentadec-6-ene, 5-methyl-8-methyl-12-ethyl-hexadec-7-ene, 5-methyl-8-methyl-12-ethyl-hexadec-8-ene, 5-ethyl-7-methyl-12-methyl-hexadec-6-ene, 5-ethyl-7-methyl-12-methyl-hexadec-7-ene, 5-methyl-8-methyl-13-methyl-heptadec-7-ene, 5-methyl-8-methyl-13-methyl-heptadec-8-ene, or combinations thereof.

In aspects, the C₂₀ trisubstituted olefin composition can contain a second group of C₂₀ trisubstituted olefins having a linear carbon chain having 16 to 18 carbon atoms, wherein the linear carbon chain has a first alkyl group on a carbon in a 5 or 9 position of the linear carbon chain, wherein the first alkyl group is a methyl group, an ethyl group, or a propyl group, wherein the linear carbon chain has a second alkyl group on a carbon in a 6, 7, 8, or 14 position of the linear carbon chain, wherein the second alkyl group is a methyl group, and wherein a carbon-carbon double bond is on a carbon in the 5, 6, 7, 8, or 9 position of the linear carbon chain.

In some aspects, the second group can contain 5-propyl-6-methyl-hexadec-5-ene, 5-propyl-6-methyl-hexadec-6-ene, 5-propyl-8-methyl-hexadec-7-ene, 5-propyl-8-methyl-hexadec-8-ene, 5-ethyl-7-methyl-heptadec-7-ene, 5-ethyl-7-methyl-heptadec-8-ene, 5-ethyl-9-methyl-heptadec-8-ene, 5-ethyl-9-methyl-heptadec-9-ene, 5-methyl-8-methyl-octadec-7-ene, 5-methyl-8-methyl-octadec-8-ene, 9-methyl-14-methyl-octadec-8-ene, 9-methyl-14-methyl-octadec-9-ene, or combinations thereof.

In aspects, the C₂₀ trisubstituted olefin composition can contain a second group of C₂₀ trisubstituted olefins, wherein the C₂₀ trisubstituted olefin has a linear carbon chain having 19 carbon atoms, wherein the linear carbon chain has a first alkyl group on a carbon in a 9 position of the linear carbon chain, wherein the first alkyl group is a methyl group, and wherein a carbon-carbon double bond is on a carbon in a 8 or 9 position of the linear carbon chain.

In some aspects, the third group can contain 9-methyl-nonadec-8-ene, 9-methyl-nonadec-9-ene, or a combination thereof.

In aspects, the C₂₀ trisubstituted olefin composition can contain any combination of the C₂₀ trisubstituted olefins of the first group, the second group, the third group, or any combination of the groups. In some aspects, C₂₀ trisubstituted olefin composition can contain 5-propyl-6-methyl-9-propyl-tridec-5-ene, 5-propyl-6-methyl-9-propyl-tridec-6-ene, 4-butyl-7-methyl-10-methyl-tetradec-6-ene, 4-butyl-7-methyl-10-methyl-tetradec-7-ene, 5-propyl-6-methyl-10-ethyl-tetradec-5-ene, 5-propyl-6-methyl-10-ethyl-tetradec-6-ene, 5-ethyl-7-methyl-10-propyl-tetradec-6-ene, 5-ethyl-7-methyl-10-propyl-tetradec-7-ene, 5-ethyl-7-methyl-11-ethyl-pentadec-6-ene, 5-ethyl-7-methyl-11-ethyl-pentadec-7-ene, 5-propyl-6-methyl-11-methyl-pentadec-5-ene, 5-propyl-6-methyl-11-methyl-pentadec-6-ene, 5-methyl-8-methyl-12-ethyl-hexadec-7-ene, 5-methyl-8-methyl-12-ethyl-hexadec-8-ene, 5-ethyl-7-methyl-12-methyl-hexadec-6-ene, 5-ethyl-7-methyl-12-methyl-hexadec-7-ene, 5-methyl-8-methyl-13-methyl-heptadec-7-ene, 5-methyl-8-methyl-13-methyl-heptadec-8-ene, 5-propyl-6-methyl-hexadec-5-ene, 5-propyl-6-methyl-hexadec-6-ene, 5-propyl-8-methyl-hexadec-7-ene, 5-propyl-8-methyl-hexadec-8-ene, 5-ethyl-7-methyl-heptadec-7-ene, 5-ethyl-7-methyl-heptadec-8-ene, 5-ethyl-9-methyl-heptadec-8-ene, 5-ethyl-9-methyl-heptadec-9-ene, 5-methyl-8-methyl-octadec-7-ene, 5-methyl-8-methyl-octadec-8-ene, 9-methyl-14-methyl-octadec-8-ene, 9-methyl-14-methyl-octadec-9-ene, 9-methyl-nonadec-8-ene, 9-methyl-nonadec-9-ene, or combinations thereof.

In aspects, the C₂₀ trisubstituted olefins are present the C₂₀ trisubstituted olefin composition in an amount of greater than 80, 81, 82, 83, 84, 85, 86, 87, 88, 89, 90, 91, 92, 93, 94, 95, 96, 97, 98, or 99 mol % based on a total moles of the C₂₀ trisubstituted olefin composition.

In additional aspects, the process of contacting the C₂₀ 2-substituted alpha olefin composition with the acidic catalyst has a conversion and selectivity to the C₂₀ trisubstituted olefins sufficient to produce the C₂₀ trisubstituted olefin composition having less than 2 or 1 mol % C₄₀ olefins based on the total moles of the C₂₀ trisubstituted olefin composition. The dimerization of the C₂₀ 2-substituted alpha olefin is minimized by performing the contacting step at a temperature described herein.

In additional aspects, the process of contacting the C₂₀ 2-substituted alpha olefin composition with the acidic catalyst has a conversion and selectivity to the C₂₀ trisubstituted olefins sufficient to produce the C₂₀ trisubstituted olefin composition having less than 10, 9, 8, 7, 6, 5, 4, 3, 2, or 1 mol % C₂₀ 2-substituted alpha olefins based on the total moles of the C₂₀ trisubstituted olefin composition.

The C₂₀ trisubstituted olefin composition can have various uses. For example, the C₂₀ trisubstituted olefin composition can be used as a feedstock for producing a paper sizing agent. For example, the C₂₀ trisubstituted olefin composition can be used as a feedstock for producing an alkenyl succinic anhydride.

### Hydrogenation

In aspects, the process can also include hydrogenating the C₂₀ trisubstituted olefin composition to form saturated C₂₀ hydrocarbons.

Hydrogenation can be accomplished by any means known to those with ordinary skill in the art with the aid of this disclosure. In aspects, all or a portion of the oligomer product can be separated from the monomer. The C₂₀ trisubstituted olefin composition can be fed to a hydrogenation unit configured to hydrogenate unsaturated double bonds of olefins in the C₂₀ trisubstituted olefin composition and to produce a polyalphaolefin product comprising the polyalphaolefins (saturated C₂₀ hydrocarbons, and saturated hydrocarbons for any other olefins in the C₂₀ trisubstituted olefin composition).

Generally, hydrogenation can include contacting the C₂₀ trisubstituted olefin composition and a hydrogenation catalyst to form a polyalphaolefin under conditions capable of hydrogenating the C₂₀ trisubstituted olefin composition. In aspects, the hydrogenation catalyst can comprise, or consist essentially of, a supported Group 7, 8, 9, and 10 metals. In some aspects, the hydrogenation catalyst can be selected from the group consisting of one or more of Ni, Pd, Pt, Co, Rh, Fe, Ru, Os, Cr, Mo, and W, supported on silica, alumina, clay, titania, zirconia, or a mixed metal oxide supports. In other aspects, the hydrogenation catalyst can be nickel supported on kieselguhr, platinum or palladium supported on alumina, or cobalt-molybdenum supported on alumina; alternatively, nickel supported on kieselguhr; alternatively, platinum or palladium supported on alumina; or alternatively, cobalt-molybdenum supported on alumina. In yet other aspects, the hydrogenation catalyst can be one or more of the group consisting of nickel supported on kieselguhr, silica, alumina, clay, or silica-alumina.

Generally, hydrogenation of the C₂₀ trisubstituted olefin composition to form a polyalphaolefin can be performed in any type of process and/or reactor which can hydrogenate the C₂₀ trisubstituted olefin composition. In an aspect, the hydrogenation of the C₂₀₊ olefin portion to form a polyalphaolefin can be performed in a batch process, a continuous process; or any combination thereof, alternatively a batch process; or alternatively a continuous process. In some aspects, the hydrogenation of the C₂₀ trisubstituted olefin composition to form a polyalphaolefin can be performed in a slurry reactor, a continuous stirred tank reactor, a fixed bed reactor or any combination thereof; alternatively, a slurry reactor; alternatively, a continuous stirred tank reactor; or alternatively, a fixed bed reactor. Generally, the polyalphaolefin product can be filtered to separate the hydrogenation catalyst and/or catalyst fines from the polyalphaolefins. Further, the polyalphaolefins can be distilled to further purify the polyalphaolefin into polyalphaolefin fractions; alternatively, distilled to form two or more compositions comprising, or consisting essentially of, polyalphaolefins having different nominal viscosities; or alternatively, distilled to further purify the polyalphaolefins and form two or more compositions comprising, or consisting essentially of, polyalphaolefins having different nominal viscosities.

The quantity of hydrogenation catalyst utilized to hydrogenate the C₂₀ trisubstituted olefin composition is dependent upon the identity of the hydrogenation catalyst and the particular hydrogenation process utilized. Generally, the amount of hydrogenation catalyst used can be any amount which can produce the desired polyalphaolefin product under the desired conditions capable of forming the polyalphaolefins. In a non-fixed bed hydrogenation process (e.g., slurry reactors or continuous stirred tank reactors, among others), the amount of hydrogenation catalyst used in the hydrogenation can range from 0.001 wt % to 20 wt %, 0.01 wt % to 15 wt %, 0.1 wt % to 10 wt %, or 1 wt % to 5 wt %. In a fixed bed process, the WHSV (weight hourly space velocity) of the C₂₀ trisubstituted olefin composition over the hydrogenation catalyst can range from 0.01 to 10, 0.05 to 7.5, or 0.1 to 5. The wt % of the hydrogenation catalyst is based upon the total weight of the hydrogenation catalyst and the oligomer product (or portion of oligomer product) being subjected to hydrogenation.

Generally, the conditions capable of hydrogenating the C₂₀ trisubstituted olefin composition can include a hydrogen pressure, a temperature, a contact time, or any combination thereof; alternatively, a hydrogen pressure and a temperature; alternatively, a hydrogen pressure, a temperature, and a contact time. In aspects, the temperature of the hydrogenation that can be utilized as a condition capable of hydrogenating the C₂₀ trisubstituted olefin composition can range from 25° C. to 350° C., from 50° C. to 300° C., from 60° C. to 250° C., or from 70° C. to 200° C. In aspects, the hydrogen pressure that can be utilized as a condition capable of hydrogenating the C₂₀ trisubstituted olefin composition can range from 100 kPa to 10 MPa, 250 kPa to 7 MPa, 500 kPa to 5 MPa, or 750 kPa to 2 MPa. In aspects, the contact time that can be utilized as a condition capable of hydrogenating the C₂₀ trisubstituted olefin composition can range from 1 minutes to 100 hours, from 2 minutes to 50 hours, 5 minutes to 25 hour, or 10 minutes to 10 hours.

In aspects, the saturated C₂₀ hydrocarbons can be used as a lubrication fluid, a hydraulic fluid, a drilling fluid, a fracturing fluid, a thermal management fluid, a metal working fluid, a coolant fluid, a dielectric coolant fluid, or a combination thereof.

### Additional Description

Aspect 1. A process comprising: contacting a C₂₀ 2-substituted alpha olefin composition with an acidic catalyst to form a C₂₀ trisubstituted olefin composition comprising a C₂₀ trisubstituted olefin. In a sub-aspect, the process can include contacting a branched C₁₀ olefin composition with a dimerization catalyst or a dimerization catalyst system to form the C₂₀ 2-substituted alpha olefin composition.

Aspect 2. The process of Aspect 1, wherein the C₂₀ trisubstituted olefin has a linear carbon chain having 13 to 17 carbon atoms, wherein the linear carbon chain has a first alkyl group on a carbon in a 4 or 5 position of the linear carbon chain, wherein the first alkyl group is a methyl group, an ethyl group, a propyl group, or a butyl group, wherein the linear carbon chain has a second alkyl group on a carbon in a 6, 7, or 8 position of the linear carbon chain, wherein the second alkyl group is a methyl group, wherein the linear carbon chain has a third alkyl group on a carbon in a 9, 10, 11, 12, or 13 position of the linear carbon chain, wherein the third alkyl group is a methyl group, an ethyl group, or a propyl group, wherein a carbon-carbon double bond is on a carbon in the 5, 6, 7, or 8 position of the linear carbon chain.

Aspect 3. The process of any one of Aspects 1 to 2, wherein the C₂₀ trisubstituted olefin has a linear carbon chain having 16 to 18 carbon atoms, wherein the linear carbon chain has a first alkyl group on a carbon in a 5 or 9 position of the linear carbon chain, wherein the first alkyl group is a methyl group, an ethyl group, or a propyl group, wherein the linear carbon chain has a second alkyl group on a carbon in a 6, 7, 8, or 14 position of the linear carbon chain, wherein the second alkyl group is a methyl group, wherein a carbon-carbon double bond is on a carbon in the 5, 6, 7, 8, or 9 position of the linear carbon chain.

Aspect 4. The process of any one of Aspects 1 to 3, wherein the C₂₀ trisubstituted olefin has a linear carbon chain having 19 carbon atoms, wherein the linear carbon chain has a first alkyl group on a carbon in a 9 position of the linear carbon chain, wherein the first alkyl group is a methyl group, wherein a carbon-carbon double bond is on a carbon in a 8 or 9 position of the linear carbon chain.

Aspect 5. The process of any one of Aspects 1 to 4, wherein the C₂₀ trisubstituted olefin composition comprises: 5-propyl-6-methyl-9-propyl-tridec-5-ene, 5-propyl-6-methyl-9-propyl-tridec-6-ene, 4-butyl-7-methyl-10-methyl-tetradec-6-ene, 4-butyl-7-methyl-10-methyl-tetradec-7-ene, 5-propyl-6-methyl-10-ethyl-tetradec-5-ene, 5-propyl-6-methyl-10-ethyl-tetradec-6-ene, 5-ethyl-7-methyl-10-propyl-tetradec-6-ene, 5-ethyl-7-methyl-10-propyl-tetradec-7-ene, 5-ethyl-7-methyl-11-ethyl-pentadec-6-ene, 5-ethyl-7-methyl-11-ethyl-pentadec-7-ene, 5-propyl-6-methyl-11-methyl-pentadec-5-ene, 5-propyl-6-methyl-11-methyl-pentadec-6-ene, 5-methyl-8-methyl-12-ethyl-hexadec-7-ene, 5-methyl-8-methyl-12-ethyl-hexadec-8-ene, 5-ethyl-7-methyl-12-methyl-hexadec-6-ene, 5-ethyl-7-methyl-12-methyl-hexadec-7-ene, 5-methyl-8-methyl-13-methyl-heptadec-7-ene, 5-methyl-8-methyl-13-methyl-heptadec-8-ene, 5-propyl-6-methyl-hexadec-5-ene, 5-propyl-6-methyl-hexadec-6-ene, 5-propyl-8-methyl-hexadec-7-ene, 5-propyl-8-methyl-hexadec-8-ene, 5-ethyl-7-methyl-heptadec-7-ene, 5-ethyl-7-methyl-heptadec-8-ene, 5-ethyl-9-methyl-heptadec-8-ene, 5-ethyl-9-methyl-heptadec-9-ene, 5-methyl-8-methyl-octadec-7-ene, 5-methyl-8-methyl-octadec-8-ene, 9-methyl-14-methyl-octadec-8-ene, 9-methyl-14-methyl-octadec-9-ene, 9-methyl-nonadec-8-ene, 9-methyl-nonadec-9-ene, or combinations thereof.

Aspect 6. The process of any one of Aspects 1 to 5, wherein the C₂₀ trisubstituted olefins are present in an amount of greater than 80 mol % based on a total moles of the C₂₀ trisubstituted olefin composition.

Aspect 7. The process of any one of Aspects 1 to 6, wherein the C₂₀ trisubstituted olefin composition comprises less than 2 mol % C₄₀ olefins based on the total moles of the C₂₀ trisubstituted olefin composition.

Aspect 8. The process of any one of Aspects 1 to 7, wherein the C₂₀ trisubstituted olefin composition comprises less than 10 mol % C₂₀ 2-substituted alpha olefins based on the total moles of the C₂₀ trisubstituted olefin composition.

Aspect 9. The process of any one of Aspects 1 to 8, wherein the branched C₁₀ olefin composition comprises branched C₁₀ olefins selected from 3-propyl-1-heptene, 4-ethyl-1-octene, 5-methyl-1-nonene, or any combination thereof.

Aspect 10. The process of any one of Aspects 1 to 9, wherein the C₂₀ 2-substituted alpha olefin composition comprises C₂₀ 2-substituted alpha olefins selected from 2-(3-methylheptyl)-7-methyl-1-undecene, 2-(4-octyl)-7-methyl-1-undecene, 2-(3-methylheptyl)-5-propyl-1-nonene, 2-(2-ethylhexyl)-7-methyl-1-undecene, 2-(3-methylheptyl)-6-ethyl-1-decene, or any combination thereof.

Aspect 11. The process of any one of Aspects 1 to 10, wherein the dimerization catalyst or the dimerization catalyst system comprises an alkylaluminum compound, a zirconium compound, or a metallocene compound.

Aspect 12. The process of Aspect 11, wherein: i) the dimerization catalyst is the alkylaluminum compound and the alkylaluminum compound consists essentially of a trialkylaluminum compound; ii) the dimerization catalyst system is the zirconium compound and an aluminoxane, wherein the zirconium compound comprises zirconium dichloride; or iii) the dimerization catalyst system is the metallocene compound and (a) an aluminoxane or (b) a non-coordinating anion and an alkylaluminum compound.

Aspect 13. The process of any one of Aspects 1 to 12, wherein the acidic catalyst comprises an acidic ion exchange resin catalyst, an acidic clay catalyst, an acidic zeolite catalyst, an acidic alumina catalyst, an acidic silicate catalyst, or combinations thereof.

Aspect 14. The process of any one of Aspects 1 to 13, wherein contacting the C₂₀ 2-substituted alpha olefin composition with the acidic catalyst is performed at a temperature in a range of from 50° C. to 70° C.

Aspect 15. The process of any one of Aspects 1 to 14, wherein the C₂₀ trisubstituted olefin composition is used as a feedstock for producing a paper sizing agent.

Aspect 16. The process of Aspect 15, wherein the paper sizing agent is an alkenyl succinic anhydride.

Aspect 17. The process of any one of Aspects 1 to 16, further comprising: hydrogenating the C₂₀ trisubstituted olefin composition to form saturated C₂₀ hydrocarbons.

Aspect 18. The process of Aspect 17, wherein the saturated C₂₀ hydrocarbons are used as a lubrication fluid, a hydraulic fluid, a drilling fluid, a fracturing fluid, a thermal management fluid, a metal working fluid, a coolant fluid, a dielectric coolant fluid, or a combination thereof.

Aspect 19. A composition comprising C₂₀ trisubstituted olefins comprising 5-propyl-6-methyl-9-propyl-tridec-5-ene, 5-propyl-6-methyl-9-propyl-tridec-6-ene, 4-butyl-7-methyl-10-methyl-tetradec-6-ene, 4-butyl-7-methyl-10-methyl-tetradec-7-ene, 5-propyl-6-methyl-10-ethyl-tetradec-5-ene, 5-propyl-6-methyl-10-ethyl-tetradec-6-ene, 5-ethyl-7-methyl-10-propyl-tetradec-6-ene, 5-ethyl-7-methyl-10-propyl-tetradec-7-ene, 5-ethyl-7-methyl-11-ethyl-pentadec-6-ene, 5-ethyl-7-methyl-11-ethyl-pentadec-7-ene, 5-propyl-6-methyl-11-methyl-pentadec-5-ene, 5-propyl-6-methyl-11-methyl-pentadec-6-ene, 5-methyl-8-methyl-12-ethyl-hexadec-7-ene, 5-methyl-8-methyl-12-ethyl-hexadec-8-ene, 5-ethyl-7-methyl-12-methyl-hexadec-6-ene, 5-ethyl-7-methyl-12-methyl-hexadec-7-ene, 5-methyl-8-methyl-13-methyl-heptadec-7-ene, 5-methyl-8-methyl-13-methyl-heptadec-8-ene, 5-propyl-6-methyl-hexadec-5-ene, 5-propyl-6-methyl-hexadec-6-ene, 5-propyl-8-methyl-hexadec-7-ene, 5-propyl-8-methyl-hexadec-8-ene, 5-ethyl-7-methyl-heptadec-7-ene, 5-ethyl-7-methyl-heptadec-8-ene, 5-ethyl-9-methyl-heptadec-8-ene, 5-ethyl-9-methyl-heptadec-9-ene, 5-methyl-8-methyl-octadec-7-ene, 5-methyl-8-methyl-octadec-8-ene, 9-methyl-14-methyl-octadec-8-ene, 9-methyl-14-methyl-octadec-9-ene, 9-methyl-nonadec-8-ene, 9-methyl-nonadec-9-ene, or combinations thereof.

Aspect 20. The composition of Aspect 19, having least 80 mol % of C₂₀ trisubstituted olefins based on a total moles of the composition.

Aspect 21. The composition of Aspect 19 or Aspect 20, further comprising less than 2 mol % C₄₀ olefins based on the total moles of the composition.

Aspect 22. The composition of any one of Aspects 19 to 21, further comprising less than 10 mol % of C₂₀ 2-substituted alpha olefins based on the total moles of composition.

Aspect 23. The composition of Aspect 22, wherein the C₂₀ 2-substituted alpha olefins are selected from 2-(3-methylheptyl)-7-methyl-1-undecene, 2-(4-octyl)-7-methyl-1-undecene, 2-(3-methylheptyl)-5-propyl-1-nonene, 2-(2-ethylhexyl)-7-methyl-1-undecene, 2-(3-methylheptyl)-6-ethyl-1-decene, or any combination thereof.

Aspect 24. A composition comprising C₂₀ trisubstituted olefins comprising a linear carbon chain having 13 to 17 carbon atoms, wherein the linear carbon chain having 13 to 17 carbon atoms has a first alkyl group on a carbon in a 4 or 5 position of the linear carbon chain, wherein the first alkyl group is a methyl group, an ethyl group, a propyl group, or a butyl group, wherein the linear carbon chain having 13 to 17 carbon atoms has a second alkyl group on a carbon in a 6, 7, or 8 position of the linear carbon chain, wherein the second alkyl group is a methyl group, wherein the linear carbon chain having 13 to 17 carbon atoms has a third alkyl group on a carbon in a 9, 10, 11, 12, or 13 position of the linear carbon chain, wherein the third alkyl group is a methyl group, an ethyl group, or a propyl group, wherein a carbon-carbon double bond is on a carbon in the 5, 6, 7, or 8 position of the linear carbon chain having 13 to 17 carbon atoms.

Aspect 25. The composition of Aspect 24, having at least 80 mol % of C₂₀ trisubstituted olefins based on a total moles of the composition.

Aspect 26. The composition of Aspect 24 or Aspect 25, wherein the C₂₀ trisubstituted olefins comprise: 5-propyl-6-methyl-9-propyl-tridec-5-ene, 5-propyl-6-methyl-9-propyl-tridec-6-ene, 4-butyl-7-methyl-10-methyl-tetradec-6-ene, 4-butyl-7-methyl-10-methyl-tetradec-7-ene, 5-propyl-6-methyl-10-ethyl-tetradec-5-ene, 5-propyl-6-methyl-10-ethyl-tetradec-6-ene, 5-ethyl-7-methyl-10-propyl-tetradec-6-ene, 5-ethyl-7-methyl-10-propyl-tetradec-7-ene, 5-ethyl-7-methyl-11-ethyl-pentadec-6-ene, 5-ethyl-7-methyl-11-ethyl-pentadec-7-ene, 5-propyl-6-methyl-11-methyl-pentadec-5-ene, 5-propyl-6-methyl-11-methyl-pentadec-6-ene, 5-methyl-8-methyl-12-ethyl-hexadec-7-ene, 5-methyl-8-methyl-12-ethyl-hexadec-8-ene, 5-ethyl-7-methyl-12-methyl-hexadec-6-ene, 5-ethyl-7-methyl-12-methyl-hexadec-7-ene, 5-methyl-8-methyl-13-methyl-heptadec-7-ene, 5-methyl-8-methyl-13-methyl-heptadec-8-ene, or combinations thereof.

Aspect 27. The composition of any one of Aspects 24 to 26, wherein the C₂₀ trisubstituted olefins further comprise a linear carbon chain having 16 to 18 carbon atoms, wherein the linear carbon chain having 16 to 18 carbon atoms has a first alkyl group on a carbon in a 5 or 9 position of the linear carbon chain, wherein the first alkyl group is a methyl group, an ethyl group, or a propyl group, wherein the linear carbon chain having 16 to 18 carbon atoms has a second alkyl group on a carbon in a 6, 7, 8, or 14 position of the linear carbon chain, wherein the second alkyl group is a methyl group, wherein a carbon-carbon double bond is on a carbon in the 5, 6, 7, 8, or 9 position of the linear carbon chain having 16 to 18 carbon atoms.

Aspect 28. The composition of Aspect 25, wherein the C₂₀ trisubstituted olefins comprise: 5-propyl-6-methyl-hexadec-5-ene, 5-propyl-6-methyl-hexadec-6-ene, 5-propyl-8-methyl-hexadec-7-ene, 5-propyl-8-methyl-hexadec-8-ene, 5-ethyl-7-methyl-heptadec-7-ene, 5-ethyl-7-methyl-heptadec-8-ene, 5-ethyl-9-methyl-heptadec-8-ene, 5-ethyl-9-methyl-heptadec-9-ene, 5-methyl-8-methyl-octadec-7-ene, 5-methyl-8-methyl-octadec-8-ene, 9-methyl-14-methyl-octadec-8-ene, 9-methyl-14-methyl-octadec-9-ene, or combinations thereof.

Aspect 29. The composition of any one of Aspects 24 to 28, wherein the C₂₀ trisubstituted olefins further comprise a linear carbon chain having 19 carbon atoms, wherein the linear carbon chain having 19 carbon atoms has a first alkyl group on a carbon in a 9 position of the linear carbon chain, wherein the first alkyl group is a methyl group, wherein a carbon-carbon double bond is on a carbon in a 8 or 9 position of the linear carbon chain having 19 carbon atoms.

Aspect 30. The composition of Aspect 29, wherein the C₂₀ trisubstituted olefins comprise: 9-methyl-nonadec-8-ene, 9-methyl-nonadec-9-ene, or a combination thereof.

Aspect 31. The composition of any one of Aspects 24 to 30, further comprising less than 2 mol % C₄₀ olefins based on the total moles of the composition.

Aspect 32. The composition of any one of Aspects 24 to 31, further comprising less than 10 mol % of C₂₀ 2-substituted alpha olefins based on the total moles of composition.

Aspect 33. The composition of Aspect 32, wherein the C₂₀ 2-substituted alpha olefins are selected from 2-(3-methylheptyl)-7-methyl-1-undecene, 2-(4-octyl)-7-methyl-1-undecene, 2-(3-methylheptyl)-5-propyl-1-nonene, 2-(2-ethylhexyl)-7-methyl-1-undecene, 2-(3-methylheptyl)-6-ethyl-1-decene, or any combination thereof.

Aspect 34. A composition comprising C₂₀ trisubstituted olefins having a linear carbon chain having 16 to 18 carbon atoms, wherein the linear carbon chain having 16 to 18 carbon atoms has a first alkyl group on a carbon in a 5 or 9 position of the linear carbon chain, wherein the first alkyl group is a methyl group, an ethyl group, or a propyl group, wherein the linear carbon chain having 16 to 18 carbon atoms has a second alkyl group on a carbon in a 6, 7, 8, or 14 position of the linear carbon chain, wherein the second alkyl group is a methyl group, wherein a carbon-carbon double bond is on a carbon in the 5, 6, 7, 8, or 9 position of the linear carbon chain having 16 to 18 carbon atoms.

Aspect 35. The composition of Aspect 34, wherein the C₂₀ trisubstituted olefins comprise: 5-propyl-6-methyl-hexadec-5-ene, 5-propyl-6-methyl-hexadec-6-ene, 5-propyl-8-methyl-hexadec-7-ene, 5-propyl-8-methyl-hexadec-8-ene, 5-ethyl-7-methyl-heptadec-7-ene, 5-ethyl-7-methyl-heptadec-8-ene, 5-ethyl-9-methyl-heptadec-8-ene, 5-ethyl-9-methyl-heptadec-9-ene, 5-methyl-8-methyl-octadec-7-ene, 5-methyl-8-methyl-octadec-8-ene, 9-methyl-14-methyl-octadec-8-ene, 9-methyl-14-methyl-octadec-9-ene, or combinations thereof.

Aspect 36. The composition of Aspect 34 or Aspect 35, wherein the C₂₀ trisubstituted olefins further comprise a linear carbon chain having 19 carbon atoms, wherein the linear carbon chain having 19 carbon atoms has a first alkyl group on a carbon in a 9 position of the linear carbon chain, wherein the first alkyl group is a methyl group, wherein a carbon-carbon double bond is on a carbon in a 8 or 9 position of the linear carbon chain having 19 carbon atoms.

Aspect 37. The composition of Aspect 36, wherein the C₂₀ trisubstituted olefins comprise: 9-methyl-nonadec-8-ene, 9-methyl-nonadec-9-ene, or a combination thereof.

Aspect 38. The composition of any one of Aspects 34 to 37, having at least 80 mol % of C₂₀ trisubstituted olefins based on a total moles of the composition.

Aspect 39. The composition of any one of Aspects 34 to 38, further comprising less than 2 mol % C₄₀ olefins based on the total moles of the composition.

Aspect 40. The composition of any one of Aspects 34 to 39, further comprising less than 10 mol % of C₂₀ 2-substituted alpha olefins based on the total moles of composition.

Aspect 41. The composition of Aspect 40, wherein the C₂₀ 2-substituted alpha olefins are selected from 2-(3-methylheptyl)-7-methyl-1-undecene, 2-(4-octyl)-7-methyl-1-undecene, 2-(3-methylheptyl)-5-propyl-1-nonene, 2-(2-ethylhexyl)-7-methyl-1-undecene, 2-(3-methylheptyl)-6-ethyl-1-decene, or any combination thereof.

Aspect 42. A composition comprising C₂₀ trisubstituted olefins having a linear carbon chain having 19 carbon atoms, wherein the linear carbon chain having 19 carbon atoms has a first alkyl group on a carbon in a 9 position of the linear carbon chain, wherein the first alkyl group is a methyl group, wherein a carbon-carbon double bond is on a carbon in a 8 or 9 position of the linear carbon chain having 19 carbon atoms.

Aspect 43. The composition of Aspect 42, wherein the C₂₀ trisubstituted olefins comprise: 9-methyl-nonadec-8-ene, 9-methyl-nonadec-9-ene, or a combination thereof.

Aspect 44. The composition of any one of Aspects 42 to 43, having at least 80 mol % of C₂₀ trisubstituted olefins based on a total moles of the composition.

Aspect 45. The composition of any one of Aspects 42 to 44, further comprising less than 2 mol % C₄₀ olefins based on the total moles of the composition.

Aspect 46. The composition of any one of Aspects 42 to 45, further comprising less than 10 mol % of C₂₀ 2-substituted alpha olefins based on the total moles of composition.

Although the present disclosure and its advantages have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A process comprising:
contacting a branched C₁₀ olefin composition with a dimerization catalyst or a dimerization catalyst system to form a C₂₀ 2-substituted alpha olefin composition; and
contacting the C₂₀ 2-substituted alpha olefin composition with an acidic catalyst to form a C₂₀ trisubstituted olefin composition comprising a C₂₀ trisubstituted olefin.

2. The process of claim 1,
wherein the C₂₀ trisubstituted olefin has a linear carbon chain having 13 to 17 carbon atoms,
wherein the linear carbon chain has a first alkyl group on a carbon in a 4 or 5 position of the linear carbon chain, wherein the first alkyl group is a methyl group, an ethyl group, a propyl group, or a butyl group,
wherein the linear carbon chain has a second alkyl group on a carbon in a 6, 7, or 8 position of the linear carbon chain, wherein the second alkyl group is a methyl group,
wherein the linear carbon chain has a third alkyl group on a carbon in a 9, 10, 11, 12, or 13 position of the linear carbon chain, wherein the third alkyl group is a methyl group, an ethyl group, or a propyl group,
wherein a carbon-carbon double bond is on a carbon in the 5, 6, 7, or 8 position of the linear carbon chain.

3. The process of claim 1,
wherein the C₂₀ trisubstituted olefin has a linear carbon chain having 16 to 18 carbon atoms,
wherein the linear carbon chain has a first alkyl group on a carbon in a 5 or 9 position of the linear carbon chain, wherein the first alkyl group is a methyl group, an ethyl group, or a propyl group,
wherein the linear carbon chain has a second alkyl group on a carbon in a 6, 7, 8, or 14 position of the linear carbon chain, wherein the second alkyl group is a methyl group,
wherein a carbon-carbon double bond is on a carbon in the 5, 6, 7, 8, or 9 position of the linear carbon chain.

4. The process of claim 1,
wherein the C₂₀ trisubstituted olefin has a linear carbon chain having 19 carbon atoms,
wherein the linear carbon chain has a first alkyl group on a carbon in a 9 position of the linear carbon chain, wherein the first alkyl group is a methyl group,
wherein a carbon-carbon double bond is on a carbon in a 8 or 9 position of the linear carbon chain.

5. The process of claim 1, wherein the C₂₀ trisubstituted olefin composition comprises:
5-propyl-6-methyl-9-propyl-tridec-5-ene, 5-propyl-6-methyl-9-propyl-tridec-6-ene, 4-butyl-7-methyl-10-methyl-tetradec-6-ene, 4-butyl-7-methyl-10-methyl-tetradec-7-ene, 5-propyl-6-methyl-10-ethyl-tetradec-5-ene, 5-propyl-6-methyl-10-ethyl-tetradec-6-ene, 5-ethyl-7-methyl-10-propyl-tetradec-6-ene, 5-ethyl-7-methyl-10-propyl-tetradec-7-ene, 5-ethyl-7-methyl-11-ethyl-pentadec-6-ene, 5-ethyl-7-methyl-11-ethyl-pentadec-7-ene, 5-propyl-6-methyl-11-methyl-pentadec-5-ene, 5-propyl-6-methyl-11-methyl-pentadec-6-ene, 5-methyl-8-methyl-12-ethyl-hexadec-7-ene, 5-methyl-8-methyl-12-ethyl-hexadec-8-ene, 5-ethyl-7-methyl-12-methyl-hexadec-6-ene, 5-ethyl-7-methyl-12-methyl-hexadec-7-ene, 5-methyl-8-methyl-13-methyl-heptadec-7-ene, 5-methyl-8-methyl-13-methyl-heptadec-8-ene,
5-propyl-6-methyl-hexadec-5-ene, 5-propyl-6-methyl-hexadec-6-ene, 5-propyl-8-methyl-hexadec-7-ene, 5-propyl-8-methyl-hexadec-8-ene, 5-ethyl-7-methyl-heptadec-7-ene, 5-ethyl-7-methyl-heptadec-8-ene, 5-ethyl-9-methyl-heptadec-8-ene, 5-ethyl-9-methyl-heptadec-9-ene, 5-methyl-8-methyl-octadec-7-ene, 5-methyl-8-methyl-octadec-8-ene, 9-methyl-14-methyl-octadec-8-ene, 9-methyl-14-methyl-octadec-9-ene,
9-methyl-nonadec-8-ene, 9-methyl-nonadec-9-ene,
or combinations thereof.

6. The process of claim 1, wherein the C₂₀ trisubstituted olefins are present in an amount of greater than 80 mol % based on a total moles of the C₂₀ trisubstituted olefin composition.

7. The process of claim 6, wherein the C₂₀ trisubstituted olefin composition comprises less than 2 mol % C₄₀ olefins based on the total moles of the C₂₀ trisubstituted olefin composition.

8. The process of claim 6, wherein the C₂₀ trisubstituted olefin composition comprises less than 10 mol % C₂₀ 2-substituted alpha olefins based on the total moles of the C₂₀ trisubstituted olefin composition.

9. The process of claim 1, wherein the branched C₁₀ olefin composition comprises branched C₁₀ olefins selected from 3-propyl-1-heptene, 4-ethyl-1-octene, 5-methyl-1-nonene, or any combination thereof.

10. The process of claim 1, wherein the C₂₀ 2-substituted alpha olefin composition comprises C₂₀ 2-substituted alpha olefins selected from 2-(3-methylheptyl)-7-methyl-1-undecene, 2-(4-octyl)-7-methyl-1-undecene, 2-(3-methylheptyl)-5-propyl-1-nonene, 2-(2-ethylhexyl)-7-methyl-1-undecene, 2-(3-methylheptyl)-6-ethyl-1-decene, or any combination thereof.

11. The process of claim 1, wherein the dimerization catalyst or the dimerization catalyst system comprises an alkylaluminum compound, a zirconium compound, or a metallocene compound.

12. The process of claim 11, wherein:
i) the dimerization catalyst is the alkylaluminum compound and the alkylaluminum compound consists essentially of a trialkylaluminum compound;
ii) the dimerization catalyst system is the zirconium compound and an aluminoxane, wherein the zirconium compound comprises zirconium dichloride; or
iii) the dimerization catalyst system is the metallocene compound and (a) an aluminoxane or (b) a non-coordinating anion and an alkylaluminum compound.

13. The process of claim 1, wherein the acidic catalyst comprises an acidic ion exchange resin catalyst, an acidic clay catalyst, an acidic zeolite catalyst, an acidic alumina catalyst, an acidic silicate catalyst, or combinations thereof.

14. The process of claim 1, wherein contacting the C₂₀ 2-substituted alpha olefin composition with the acidic catalyst is performed at a temperature in a range of from 50° C. to 70° C.

15. The process of claim 1, wherein the C₂₀ trisubstituted olefin composition is used as a feedstock for producing a paper sizing agent.

16. The process of claim 15, wherein the paper sizing agent is an alkenyl succinic anhydride.

17. The process of claim 1, further comprising:
hydrogenating the C₂₀ trisubstituted olefin composition to form saturated C₂₀ hydrocarbons.

18. The process of claim 17, wherein the saturated C₂₀ hydrocarbons are used as a lubrication fluid, a hydraulic fluid, a drilling fluid, a fracturing fluid, a thermal management fluid, a metal working fluid, a coolant fluid, a dielectric coolant fluid, or a combination thereof.

19. A composition comprising at least 80 mol % of C₂₀ trisubstituted olefins based on a total moles of the composition, wherein the C₂₀ trisubstituted olefins comprise
5-propyl-6-methyl-9-propyl-tridec-5-ene, 5-propyl-6-methyl-9-propyl-tridec-6-ene, 4-butyl-7-methyl-10-methyl-tetradec-6-ene, 4-butyl-7-methyl-10-methyl-tetradec-7-ene, 5-propyl-6-methyl-10-ethyl-tetradec-5-ene, 5-propyl-6-methyl-10-ethyl-tetradec-6-ene, 5-ethyl-7-methyl-10-propyl-tetradec-6-ene, 5-ethyl-7-methyl-10-propyl-tetradec-7-ene, 5-ethyl-7-methyl-11-ethyl-pentadec-6-ene, 5-ethyl-7-methyl-11-ethyl-pentadec-7-ene, 5-propyl-6-methyl-11-methyl-pentadec-5-ene, 5-propyl-6-methyl-11-methyl-pentadec-6-ene, 5-methyl-8-methyl-12-ethyl-hexadec-7-ene, 5-methyl-8-methyl-12-ethyl-hexadec-8-ene, 5-ethyl-7-methyl-12-methyl-hexadec-6-ene, 5-ethyl-7-methyl-12-methyl-hexadec-7-ene, 5-methyl-8-methyl-13-methyl-heptadec-7-ene, 5-methyl-8-methyl-13-methyl-heptadec-8-ene,
5-propyl-6-methyl-hexadec-5-ene, 5-propyl-6-methyl-hexadec-6-ene, 5-propyl-8-methyl-hexadec-7-ene, 5-propyl-8-methyl-hexadec-8-ene, 5-ethyl-7-methyl-heptadec-7-ene, 5-ethyl-7-methyl-heptadec-8-ene, 5-ethyl-9-methyl-heptadec-8-ene, 5-ethyl-9-methyl-heptadec-9-ene, 5-methyl-8-methyl-octadec-7-ene, 5-methyl-8-methyl-octadec-8-ene, 9-methyl-14-methyl-octadec-8-ene, 9-methyl-14-methyl-octadec-9-ene,
9-methyl-nonadec-8-ene, 9-methyl-nonadec-9-ene,
or combinations thereof.

20. The composition of claim 19, further comprising less than 2 mol % C₄₀ olefins based on the total moles of the composition and less than 10 mol % of C₂₀ 2-substituted alpha olefins based on the total moles of composition.
